(19) United States
(12) Patent Application Publication
(10) Pub. No.: US 2024/0002927 A1
(43) Pub. Date: Jan. 4, 2024

(54) METHODS FOR DETECTION OF NUCLEOTIDE MODIFICATION

(71) Applicant: BLUECAT IP LIMITED, Cambridge (GB)

(72) Inventors: Shankar BALASUBRAMANIAN, Cambridge (GB); Ben MORTISHIRE-SMITH, Cambridge (GB); Sidney BECKER, Cambridge (GB)

(21) Appl. No.: 18/313,146

(22) Filed: May 5, 2023

Related U.S. Application Data
(63) Continuation of application No. PCT/EP2021/081159, filed on Nov. 9, 2021.

(30) Foreign Application Priority Data
Nov. 9, 2020 (GB) 2017653.3

(51) Int. Cl. C12Q 1/6869; C12Q 1/6858

(52) U.S. Cl. CPC C12Q 1/6869; C12Q 1/6858; C12Q 1/6806

(57) ABSTRACT

The invention provides a method of identifying a modified cytosine residue in a polynucleotide. The method includes the steps of reducing and deaminating a 5-methylcytosine residue within the polynucleotide and sequencing the polynucleotide, such that the location of the modified cytosine residue can be identified. The invention also provides a method of identifying a 5-methylcytosine residue, a 5-carboxylcytosine residue or a nucleotide in a polynucleotide. The method involves treating the polynucleotide with a radical initiator. The presence of the 5-methylcytosine residue, 5-carboxylcytosine residue or nucleotide can be identified as a residue such as thymine detected by sequencing of the treated polynucleotide. Also provided is a method of converting a 5-methylcytosine residue in a polynucleotide to a dihydrothymine residue, and use of a radical initiator to convert a 5-methylcytosine residue in a polynucleotide to a dihydrothymine residue, or to convert a 5-carboxylcytosine residue to a dihydrouracil residue.

### RELATED APPLICATION

The present application is a continuation of International Application No. PCT/EP2021/081159 filed 9 Nov. 2021, which claims priority to and the benefit of GB 2017653.3 filed on 9 Nov. 2020 (09/11/2020), the contents of which are hereby incorporated by reference in their entirety.

The project leading to this application has received funding from the European Union's Horizon 2020 research and innovation program under the Marie Sklodowska-Curie grant agreement no. 887491.

### FIELD OF THE INVENTION

This invention relates to the detection of modified cytosine residues and, in particular, to the sequencing of nucleic acids that contain modified cytosine residues. In particular, the present invention relates to a method of detecting a nucleoside or a nucleotide sequence containing 5-methylcytosine (5mC) or 5-carboxylcytosine (5caC).

### BACKGROUND

5-Methylcytosine (5mC) is an epigenetic DNA mark that plays important roles in gene silencing and genome stability, and is found enriched at CpG dinucleotides (Deaton et al.). 5-Hydroxymethylcytosine (5hmC) has been proposed as an intermediate in active DNA demethylation, for example by deamination or via further enzymatic oxidation of 5hmC to 5-formylcytosine (5fC) and 5-carboxycytosine (5caC), followed by base excision repair. However, 5hmC may also constitute an epigenetic mark per se.

Methylation of cytosine is the most abundant DNA modification and exerts a variety of repressive or activating effects on gene expression depending on genomic region and sequence context. Sequencing methodologies based on this chemistry may have applications in the analysis of epigenetic modifications in genomic DNA. These technologies can be used to investigate how methylation contributes to changes in gene expression during embryonic development and how epigenetic dysregulation is linked to the development of cancer. A deeper understanding of the role of DNA base modifications may reveal new opportunities for therapeutic intervention, and also have diagnostic potential for the early detection of diseases caused by epigenetic changes.

It is possible to detect and quantify the level of 5mC and 5hmC present in total genomic DNA by analytical methods that include, most notably, bisulfite sequencing. However, bisulfite sequencing alone does not distinguish between 5mC and 5hmC, and alternative strategies are required to achieve a discrimination between these two modified residues.

One approach for sequencing DNA methylation (5mC) uses the bisulfite conversion, where a C to U change is effected in a nucleotide sequence, which change is then read as T in the subsequent amplification and sequencing.

Limitations of this approach include the reduction of the genetic sequence of each DNA strand to essentially three letters instead of four, which makes it challenging to detect genetic variants: for example all Cs convert to Ts in the sequencing, which makes it impossible to detect C-to-T genetic variants (the most common mutation). Also, bisulfite conversion reduces the complexity of the sequence making it computationally challenging to accurately re-align sequenced reads to the reference genome. Lastly, bisulfite is known to cause some cleavage of DNA at C residues which can cause loss of sequenceable material.

Other alternative methods for detection of 5-methylcytosine (5mC) and 5-hydroxymethylcytosine (5hmC) at base resolution include where a 5mC residue within a nucleotide sequence is converted to 5caC in one step. In a subsequent second distinct step the 5caC residue is converted to dihydrouracil (DHU) under the action of a borane-containing compound. Here, the formation of the dihydrouracil involves the reduction of the C5-C6 bond, accompanied by decarboxylation of the 5-carboxy group. Subsequent amplification of the nucleotide sequence can convert DHU to thymine, enabling a C-to-T transition of 5mC.

The oxidation and reduction reactions constitute two independent steps, with the requirement for the purification of the 5caC-containing nucleotide sequence prior to its subsequent conversion to DHU. These multiple steps may complicate methods of detecting a 5mC residue within a nucleotide sequence and also make it difficult to integrate the process into automated sequencing methods: additional programming is required to accommodate the two reaction steps, and their associated work-up procedures. The two-step sequence may also reduce the amount of sample nucleotide sequence that is available for sequencing and increase sample recovery loss over the two steps. The use of borane-containing compounds may carry a flammability risk and/or toxicity risk.

### SEQUENCE LISTING

The instant application contains a Sequence Listing which has been submitted electronically in XML format and is hereby incorporated by reference in its entirety. Said XML copy, created on Jun. 5, 2023, is named 47224_743_301.xml and is 50,744 bytes in size.

### SUMMARY OF THE INVENTION

In a general aspect the present invention provides a method for generating a dihydrothymine (DHT) or a dihydrouracil (DHU) residue from a nucleoside or a polynucleotide containing 5-methylcytosine (5mC) or 5-carboxylcytosine (5caC) respectively.

A DHT residue may be generated directly from the corresponding 5mC residue. Thus, the reaction is performed in one step, and without the need for the preparation and/or isolation of any intermediate material, for example 5caC. The transformation may therefore be regarded as a one-pot reaction.

The transformation of the 5mC to DHT may be achieved by treatment of a nucleoside or a polynucleotide with a radical initiator optionally together with a nucleophile.

Advantageously, the conditions for the direct preparation of DHT from 5mC are also suitable for the conversion of 5caC to DHU. Thus, where a nucleoside or a polynucleotide from a sample nucleotide sequence acid contains 5caC, this may be readily converted to the DHU form. The methods of the invention may therefore be incorporated into known sequencing methods, where the preparation of 5caC, and its conversion to DHU, are key steps.

Accordingly, the present inventors have devised methods that allow modified cytosine residues, such as 5-methylcytosine (5mC) and 5-carboxylcytosine (5caC), to be distinguished from cytosine (C) at a single nucleotide resolution. These methods are applicable to all sequencing platforms and may be useful, for example, in the analysis of genomic DNA and/or of RNA.

The chemistry described in the present case allows for specific conversion of 5mC and 5caC. These methods overcome many of the limitations associated with the bisulfite conversion and the borane conversion, and therefore have great utility for applications of sequencing in research and in clinical diagnostics. The methods of the present case provide a process for preparing DHT from 5mC that is simpler than the method reported in the art for the conversion of 5mC to DHU. The methods of the present case also provide a process for preparing DHU from 5caC. These processes may also be higher yielding and quicker than the known methods for preparing DHU residues within a nucleotide sequence.

Additionally, the reactivity of other methylated C forms, such as 5-hydroxymethylcytosine (5hmC) and 5-formylcytosine (5fC), may be less than 5mC and 5caC under the reaction conditions required to convert 5mC to DHT and 5caC to DHU. The reactivity of other residues, including A, T and G may also be less than 5mC and 5caC under the reaction conditions.

The present invention provides a method for directly converting a 5-methylcytosine (5mC) residue in a polynucleotide to a dihydrothymine (DHT) residue. The method is performed without isolation of any intermediates, and it is performed in one pot.

The method is a one-step process. In this method, only one set of reagents is needed. There is no need for the isolation and purification of an intermediate nucleotide sequence. For example, there is no requirement to prepare and isolate an intermediate nucleotide sequence containing 5-carboxylcytosine (5caC).

The methods of the invention can therefore effectively bring about a C to T transformation in a polynucleotide for the residues 5mC and 5caC. A comparison between a polynucleotide from a sample prepared by the methods of the invention, and an untreated polynucleotide from the sample, will reveal a change that is associated with the presence of a modified cytosine, and specifically 5mC and/or 5caC. Each change between the treated and untreated polynucleotides can be identified at a single nucleotide level.

In a first aspect of the invention there is provided a method of transforming a 5-methylcytosine (5mC) to a dihydrothymine (DHT) in one step. The 5mC may be a nucleoside or it may be a residue within a polynucleotide. The transformation may be a radical-mediated transformation, proceeding via a radical intermediate.

The reaction is performed in the absence of a borane compound.

The invention also includes a method of identifying a modified cytosine residue in a sample nucleotide sequence, the method comprising;
(i) providing a population of polynucleotides which comprise the sample nucleotide sequence,
(ii) reducing and deaminating the population,
(iii) sequencing the polynucleotides in the population following step (ii) to produce a treated nucleotide sequence, and;
(iv) identifying the residue in the treated nucleotide sequence which corresponds to a modified cytosine residue in the sample nucleotide sequence.

The method may be used for the reduction and deamination of 5mC and/or 5caC residues within the polynucleotides, and preferably for 5mC residues.

In step (ii) the reduction may refer to the formal reduction (or saturation) of the C5-C6 bond in a modified cytosine. In step (ii) the deamination may refer to the formal loss of the amino group at the C4 position in a modified cytosine. Here, the amino group may be formally replaced with hydroxyl. Step (ii) is one step, and does not include the isolation of any intermediate compound.

In some cases, step (iii) includes sequencing the polynucleotides in the population or derivatives thereof following step (ii) to produce a treated nucleotide sequence. In step (iii) the derivatives of polynucleotides may include products of the reduction and deamination reaction in step (ii). In step (iii) the polynucleotides may include products derived after further processing of the reduced and deaminated polynucleotides obtained in step (ii).

In step (iv) the presence of a thymine residue in the treated nucleotide sequence is indicative that the modified cytosine residue in the sample nucleotide sequence is 5-methylcytosine (5mC) or 5-carboxylcytosine (5caC). Here, the thymine residue is a residue that is read as cytosine in the sequencing of an untreated population of the sample.

The methods of the present case allow for the identification of 5-methylcytosine (5mC), by conversion of this residue to dihydrothymine (DHT). Here, step (ii) is the reducing and deaminating of a 5-methylcytosine in the sample nucleotide. The method does not include the step of oxidising the 5-methyl group of the 5-methylcytosine, or the preparation or isolation of 5-carboxylcytosine as an intermediate in the reduction and deamination step.

The present invention provides a method of identifying 5-methylcytosine (5mC) or 5-carboxylcytosine (5caC) in a sample nucleotide sequence, the method comprising:
(i) providing a population of polynucleotides which comprise the sample nucleotide sequence,
(ii) treating the population with a radical initiator optionally together with a nucleophile,
(iii) sequencing the polynucleotides in the population following step (ii) to produce a treated nucleotide sequence, and;
(iv) identifying the residue in the treated nucleotide sequence which corresponds to a modified cytosine residue in the sample nucleotide sequence,
wherein the presence of a thymine residue in the treated nucleotide sequence is indicative that the modified cytosine residue in the sample nucleotide sequence is 5-methylcytosine (5mC) or 5-carboxylcytosine (5caC).

In some cases, step (iii) includes sequencing the polynucleotides in the population or derivatives thereof following step (ii) to produce a treated nucleotide sequence. In step (iii) the derivatives of polynucleotides may include products after treatment with the radical initiator optionally together with the nucleophile. The derivatives may include products derived after further processing of the treated polynucleotides obtained in step (ii).

In a preferred embodiment, the sample nucleotide includes a 5-methylcytosine (5mC) residue, and the treated nucleotide sequence includes a dihydrothymine (DHT) residue, which is derived from the 5-methylcytosine (5mC). Here, the DHT residue is produced in one step and one pot from the 5-methylcytosine (5mC).

In an alternative embodiment, the sample nucleotide includes a 5-carboxylcytosine (5caC) residue, and the treated nucleotide sequence includes a dihydrouracil (DHU) residue, which is derived from the 5-carboxylcytosine (5caC).

The invention also includes a method of identifying a modified cytosine residue in a sample nucleotide sequence, the method comprising:
(i) providing a population of polynucleotides which comprise the sample nucleotide sequence,
(ii) oxidising a first portion of said population,
(iii) treating the oxidised first portion of said population with a radical initiator optionally together with a nucleophile,
(iv) sequencing the polynucleotides in the first portion of the population following steps ii) and iii) to produce a first nucleotide sequence, and;
(v) identifying the residue in the first nucleotide sequence which corresponds to a modified cytosine residue in the sample nucleotide sequence.

Here, a residue identified in the first nucleotide sequence is indicative of a modified cytosine at the corresponding position in the sample nucleotide sequence. The modified cytosine may be 5-methylcytosine (5mC).

The product of the oxidising step (ii) may be 5caC, which may be formed from a 5mC contained in the nucleotide sequence.

In some cases, step (iv) includes sequencing the polynucleotides in the first portion of the population or derivatives thereof following steps ii) and iii) to produce a first nucleotide sequence. In step (iv) the derivatives of polynucleotides may include products after oxidation and/or treatment of the oxidation products. The derivatives may include products derived after further processing of the oxidation products and/or treated polynucleotides obtained in steps (ii) and (iii).

The oxidation step (ii) may be repeated to maximise the yield of the oxidative product.

The oxidation is may be enzymatic oxidation, such as oxidation by an oxidase, such as an oxidation a ten-eleven-translocation (TET) oxygenase, for example an oxygenase selected from TET1, TET2 and TET3.

The oxidation of the first portion in step (ii) may be an oxidation to give a 5caC residue, for example from a 5mC residue. Step (iii) may then convert the 5caC residue to a DHU residue. This gives rise to a C to T change in any subsequent amplification and sequencing.

In yet a further aspect of the invention there is provided a method for identifying a reaction condition for the transformation of a 5-methylcytosine (5mC) to a dihydrothymine (DHT), the method comprising the steps of
(i) treating a 5-methylcytosine (5mC) with one or more test reagents; and
(ii) detecting the presence of dihydrothymine (DHT) as a product of the treatment.

The treatment is performed in one-pot.

The 5mC may be a nucleoside or a residue within a polynucleotide.

The method may also include the step of treating a 5caC with the one or more test reagents, and subsequently detecting the presence of dihydrouracil (DHU) as a product of the treatment. The 5caC may be a nucleoside or a residue in a polynucleotide.

In a further aspect the invention provides a method of identifying a nucleotide in a sample nucleotide sequence, the method comprising:
(i) providing a population of polynucleotides which comprise the sample nucleotide sequence,
(ii) treating the population with a radical initiator optionally together with a nucleophile to produce treated polynucleotides,
(iii) sequencing the treated polynucleotides following (ii) to obtain a nucleotide sequence comprising a transformed nucleotide corresponding to the nucleotide in the sample nucleotide sequence, and;
(iv) identifying the transformed nucleotide in the nucleotide sequence, thereby identifying the nucleotide in the sample nucleotide sequence.

In some cases, step (iii) includes sequencing the treated polynucleotides following (ii), or derivatives thereof to obtain a nucleotide sequence comprising a transformed nucleotide corresponding to the nucleotide in the sample nucleotide sequence. In step (iii) the derivatives may include products after treatment with the radical initiator optionally together with the nucleophile. The derivatives may include products derived after further processing of the treated polynucleotides obtained in step (ii).

The transformed nucleotide may comprise a thymine residue. The nucleotide identified in the sample nucleotide sequence may corresponds to an adenine residue, a guanine residue or a cytosine residue. The nucleotide identified in the sample nucleotide sequence may corresponds to a modified cytosine residue, such as 5caC or 5mC.

These and other aspects and embodiments of the invention are described in further detail in the detailed description of the invention.

### SUMMARY OF THE FIGURES

The present invention is described with reference to the figures listed below.

FIG. 1 is a schematic showing the general methods for detecting 5mC, 5hmC and 5fC via a 5caC conversion and a DHU product. C-to-T transitions are induced at locations containing the 5caC modification, allowing it to be differentiated from canonical C within DNA. The methods of the invention may be adapted to sequencing other cytosine derivatives, for example, with the integration of prior chemoenzymatic steps. In the methods of the invention the conversion of 5caC to DHU may be performed with a radical initiator optionally together with a nucleophile, such as a transition metal photocatalyst together with a thiol.

FIG. 2 is a schematic showing the general method of the invention for detecting 5mC. Here, the 5mC is converted directly to DHT, thereby enabling direct deamination, detection and sequencing of cytosine methylation. Here, the conversion of 5mC to DHT may be performed with a radical initiator optionally together with a nucleophile, such as a transition metal photocatalyst together with a thiol.

FIG. 3 shows the proportion of bases detected from sequencing reads at cytosine positions for 5caC Oligomer 4 after photochemical treatment.

FIG. 4 shows the proportion of bases detected from sequencing reads at cytosine positions for untreated 5caC Oligomer 4.

FIG. 5 is an IGV visualization of bases detected in sequencing reads for 5caC Oligomer 4 following treatment compared to control Oligomer 5.

FIG. 6 is a graph showing the proportion of CpG sites in each modification state in a control library, TET-assisted bisulfite sequencing library, and sequencing libraries prepared using a two-step method of the present invention involving TET oxidation and photochemical treatment. In Libraries 1 & 2, the second bar indicates the CpG sites protected from deamination & determined to be methylated. In Libraries 3 to 9, the last bar indicates the rate of photochemical C-to-T conversion at CpG sites.

FIG. 7 is a graph illustrating the absolute rate at which CpG sites in different contexts were detected as methylated using an embodiment of the present invention.

FIG. 8 is a graph showing the (normalised) step efficiency with which oxidised 5mCs were converted to T by the photochemistry of the invention.

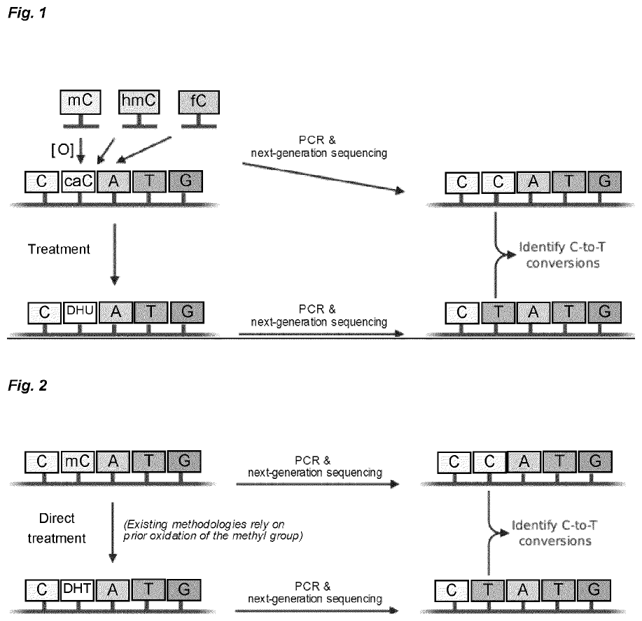

### DETAILED DESCRIPTION OF THE INVENTION

The present invention provides a method for detecting 5mC in a polynucleotide. The distinguishing feature of this method is the direct conversion of a 5mC residue within the polynucleotide to a DHT residue. This conversion formally involves C5-C6 reduction and C4 deamination, where the amino group is replaced with hydroxyl.

The reaction conditions for the direct conversion of 5mC to DHT are beneficially also suitable for converting 5caC to DHU in a polynucleotide. Thus, the methods of the invention may also be used for detecting 5caC in a polynucleotide. Such a conversion formally involves C5-C6 reduction, C5 decarboxylation and C4 deamination, where the amino group is replaced with hydroxyl.

Where a hydroxyl group replaces an amino group, as described above, it is understood that this hydroxyl group tautomerises to give the preferred keto form, as observed in the DHT and DHU residues.

Exemplary transformations are shown in the worked examples for nucleoside and polynucleotide samples (see also Scheme 1).

The present inventors have established a one-step procedure for generating a DHT residue from a 5mC residue contained within a polynucleotide and as a nucleoside.

Advantageously, the methods of this invention may also be employed to convert a 5caC residue to a DHU residue within a nucleotide sequence and as a nucleoside and may also be provided as an alternative to methods for the generation of DHU from 5caC using borane-based reagents.

The inventors have established that the conversion of 5caC to DHU works well when this residue is in its nucleoside form, and when it is present as a nucleotide within a nucleotide sequence.

For example, the worked examples in the present case show that DHU may be generated from a 5caC residue within a nucleotide sequence in essentially quantitative yields (>95%) in 15 minutes at ambient temperature. In some cases, in the present case DHU may be generated from a 5caC residue within a nucleotide sequence at >95%, >96%, >97%, >98%, or >99% in 15 minutes at ambient temperature. In some cases, in the present case DHU may be generated from a 5caC residue within a nucleotide sequence at >95%, >96%, >97%, >98%, or >99% in 10 minutes or less at ambient temperature. In some cases, in the present case DHU may be generated from a 5caC residue within a nucleotide sequence at >95%, >96%, >97%, >98%, or >99% in 5 minutes or less at ambient temperature. In some cases, in the present case DHU may be generated from a 5caC residue within a nucleotide sequence at >95%, >96%, >97%, >98%, or >99% in 2 minutes or less at ambient temperature.

The worked examples also show the conversion of 5mC and 5caC nucleosides to the respective DHT and DHU forms. In some cases, the conversion is also very high (>95%) after 4 to 6 hours at ambient temperature. In some cases, the conversion is >98% after 4 to 6 hours at ambient temperature. In some cases, the conversion is >99% after 4 to 6 hours at ambient temperature. In some cases, the conversion is >95% after less than 3 hours at ambient temperature. In some cases, the conversion is >95% after less than 2 hours at ambient temperature. In some cases, the conversion is >95% after less than 1 hour at ambient temperature.

The inventors have also established that 5mC in its nucleoside form may be converted to the corresponding DHT nucleoside form in essentially quantitative yields (>95%) in 6 hours at ambient temperature. In some cases, 5mC in its nucleoside form may be converted to the corresponding DHT nucleoside form in essentially quantitative yields (>95%) in less than 6 hours at ambient temperature. In some cases, 5mC in its nucleoside form may be converted to the corresponding DHT nucleoside form in essentially quantitative yields (>95%) in less than 5 hours at ambient temperature. In some cases, 5mC in its nucleoside form may be converted to the corresponding DHT nucleoside form in essentially quantitative yields (>95%) in less than 4 hours at ambient temperature. In some cases, 5mC in its nucleoside form may be converted to the corresponding DHT nucleoside form in essentially quantitative yields (>95%) in less than 3 hours at ambient temperature. In some cases, 5mC in its nucleoside form may be converted to the corresponding DHT nucleoside form in essentially quantitative yields (>95%) in less than 2 hours at ambient temperature. In some cases, 5mC in its nucleoside form may be converted to the corresponding DHT nucleoside form in essentially quantitative yields (>95%) in less than 1 hour at ambient temperature.

The reaction conditions in the present case are capable of converting both 5mC and 5caC to the respective DHT and DHU forms.

In some instances, 5hmC and 5fC residues are less reactive than 5mC and 5caC under the reaction conditions required to convert 5mC to DHT and 5caC to DHU. In some cases, 5hmC and 5fC residues are present at significantly lower amounts compared to 5mC. In some cases, the amount of each these residues can be determined independently from 5mC, and their presence may be accounted for. It is also possible to protect 5hmC and 5fC residues prior to any conversion of 5mC to 5caC.

In some instances, the methods of the present case allow for the formation of DHU from 5caC involving formal demethylation of the C5 methyl group, as well as C4 deamination and C5-C6 reduction under aqueous conditions. The formation of DHT from 5mC can involve C4 deamination and C5-C6 reduction under aqueous conditions. In both cases the 4-amino group is replaced with hydroxyl.

In some instances, the methods of the present case utilise radical chemistry in the reaction of the 5mC and 5caC residues. In some cases, a radical initiator is used. In some cases, this process in the present case proceeds, in part, via a radical mechanism. Thus, the methods of the present case may proceed via a radical intermediate.

Reference herein to 5mC, 5caC, DHT and DHU, and others, is typically a reference to the deoxyribonucleoside form within a polynucleotide. However, the invention also relates to other nucleotide forms, as described further below, including nucleosides.

### Radical Initiator

The methods of the present invention involve the formation of DHT from 5mC and DHU from 5caC. In some instances, the methods of the invention proceed via a radical intermediate.

The methods of the present case therefore provide for the use of a radical initiator to generate radical reactive species for the reaction of the 5mC or 5caC. The radical initiator is optionally used together with a nucleophile, and preferably it is used so.

The radical initiator may be a photo-, thermal-, or microwave-initiated radical initiator.

The radical initiator may be present at a stoichiometric amount or at an amount that is less than a stoichiometric amount. The radical initiator may also be used a catalyst, which is regenerated during the radical reaction. Here, the catalyst is preferably used in a catalytic amount, which is less than a stoichiometric amount.

In some instances, the radical initiator is used to generate radicals in the presence of a modified cytosine, such as 5mC and 5caC, optionally in the presence of the nucleophile. In some instances, the radical initiator is able to initiate the reduction and deamination process for the formation of DHT from 5mC. In some instances, the radical initiator is able to initiate the reduction, deamination and decarboxylation process for the formation of DHU from 5caC. In some instances, these reaction processes are initiated under aqueous, such as acidic aqueous, conditions.

The radical initiator is suitable for use under aqueous conditions.

Preferably, the radical initiator is photoinitiated, and is preferably active under incident visible light.

Example radical initiators include peroxides, disulfides, and azo-initiators.

The radical initiator may be a catalyst such as a single electron transfer catalyst. The catalyst may be a transition metal-based catalyst, such as Ir and Ru-based catalysts as described in further detail below, or an organic photosensitisers catalyst, such as Norrish Type I/II initiators.

The radical initiator may also be selected from a metal inorganic photocatalysts. Suitable photocatalysts may include phosphotungstic acid and iron-sulphur clusters, which may generate hydrogen disulfides or iron disulfides in acidic conditions, for example.

The radical initiator may also be an enzymatic radical initiator. An example of such includes horseradish peroxidase, such as those described by Danielson et al. An enzymatic radical initiator may also a ribonucleotide reductase or thioredoxin.

A photocatalyst is a species that is capable of absorbing light to generate an electron-hole pair (an excited state). In the present case, a single electron transfer (SET) between a species in the reaction mixture and the photocatalyst may generate an electrophilic radical cation. There may be a transfer between the photocatalyst and a modified cytosine, and/or there may be a transfer between the photocatalyst and the nucleophile.

Preferably, the photocatalyst is a visible-light photocatalyst. That is, a photocatalyst which absorbs light in the visible range to form an excited state. This avoids the need to use ultraviolet (UV) light to excite the photocatalyst. UV light may damage or degrade nucleic acids such as RNA or DNA, which would be detrimental to the methods of the present case.

Preferably, the absorption maximum for the photocatalyst is in the range 400 to 600 nm, more preferably 400 to 500 nm, and even more preferably in the range 400 to 450 nm.

The photocatalyst may be an organic photocatalyst or a transition metal photocatalyst.

Examples of organic photocatalysts are those based on acridinium, pyrylium, phenothiazine, phenoxazine, phenazine, phthalonitrile or flavin ring systems. Specific examples include triphenylpyrylium, 9-Mesityl-10-methylacridinium (Mes-Acr), Eosin Y, Fluorescein, riboflavin, riboflavin tetrabutyrate, riboflavin monophosphate and flavin adenine dinucleotide

Preferably, the photocatalyst is a transition metal photocatalyst.

Transition metal photocatalysts typically comprise one or more ligands. The ligands may be any ligand that is suitable for stabilizing the metal in the transition metal photocatalyst. Where two or more ligands are present, the ligands may be identical (homoleptic) or different (heteroleptic).

Example ligands for transition metal photocatalysts include those based on bipyridine ring systems, phenylpyridine ring systems, bipyrimidine ring systems, bipyrazine ring systems, phenanthroline ring systems and triphenylene ring systems.

Each ligand ring system may be substituted or unsubstituted. Typically substitutions include C₁₋₆ alkyl, C₁₋₃ haloalkyl, halo, and C₁₋₃ alkoxy.

Examples of phenylpyridine ligands include 2-phenylpyridine (ppy), 2-(4-fluorophenyl) pyridine (p-Fppy), 2-(4-trifluoromethylphenyl)pyridine (p-CF₃ppy), 4-tertbutyl-2-(4-fluorophenyl)pyridine (p-F(tBu)ppy), 2-(2,4-difluorophenyl)pyridine (dFppy), 4-tertbutyl-2-(2,4-difluorophenyl)pyridine (dF(t-Bu)ppy), 2-(2,4-difluorophenyl)-5-(trifluoromethyl)pyridine (dF(CF₃)ppy), 2-(2,4-difluorophenyl)-5-fluoro-pyridine (dF(F)ppy), 2-(2,4-difluorophenyl)-5-methyl-pyridine (dF(Me)ppy), 2-(2,4-difluorophenyl)-5-methoxy-pyridine (dF(OMe)ppy), 2-(2-fluoro-4-(trifluoromethyl)phenyl)-5-(trifluoromethyl)pyridine (FCF₃(CF₃)ppy), 4-methyl-2-(p-tolyl)pyridine (Me(Me)ppy) and 2-(4-fluorophenyl)-5-methyl-pyridine (p-F(Me)ppy).

Examples of bipyridine ligands include 2,2-bipyridine (bpy), 4,4′-dimethyl-2,2′-bipyridine (dmbpy), 4,4′-di-tertbutyl-2,2′-bipyridine (dtbbpy), 4,4′-bis(trifluoromethyl)-2,2′-bipyridine (4,4′-dCF₃bpy), 5,5′-bis(trifluoromethyl)-2,2′-bipyridine (5,5′-dCF₃bpy).

Examples of phenylpyridine ligands include 2-(2,4-difluorophenyl)-5-fluoropyridine, 2-(2,4-difluorophenyl)-5-methoxypyridine, 2-(2,4-difluorophenyl)-5-methylpyridine, 2-(2,4-difluorophenyl)-5-(trifluoromethyl)pyridine, 2-(4-fluorophenyl)-5-methylpyridine and 2-[2-Fluoro-4-(trifluoromethyl)phenyl]-5-(trifluoromethyl)pyridine.

Examples of bipyrimidine ligands include 2,2′-bipyrimide (bpm),

Examples of bipyrazine ligands include 2,2′-bipyrazine (bpz).

Examples of phenanthroline ligands include 1,10-phenanthroline (phen), 1,4,5,8-tetraazaphenanthrene (tap) and dipyridophenazine (dppz).

Examples of triphenylene ligands include 1,4,5,8,9,12-hexaazatriphenylene (hat).

Examples of transition metal photocatalysts are those comprising ruthenium (Ru) or iridium (Ir).

Specific examples of ruthenium photocatalysts include [Ru(bpy)₃]²⁺, [Ru(phen)₃]²⁺, [Ru(bpm)₃]²⁺, [Ru(bpz)₃]²⁺, [Ru(4,4′-dCF₃bpy)₃]²⁺, [Ru(dmbpy)₃]²⁺ and [Ru(dtbbpy)₃]²⁺.

Examples of iridium photocatalysts include [Ir(dF(CF₃)ppy)₂(dtbpy)]⁺, [Ir(ppy)₃], [Ir(dFppy)₃], [Ir(p-Fppy)₃], [Ir(p-F(Me)ppy)₂(dtbbpy)]⁺, [Ir(Me(Me)ppy)₂(dtbbpy)]⁺, [Ir(FCF₃(CF₃)ppy)₂(dtbbpy)]⁺, [Ir(ppy)₂(dtbbpy)]⁺, [Ir(dFppy)₂(dtbbpy)]⁺, [Ir(dF(Me)ppy)₂(dtbbpy)]⁺, [Ir(dF(Me)ppy)₂(4,4′-dCF₃bpy)]⁺, [Ir(dF(F)ppy)₂(dtbbpy)]⁺.

Preferably, the transition metal photocatalyst is an iridium photocatalyst.

More preferably, the transition metal photocatalyst is[Ir(dF(CF₃)ppy)₂(dtbpy)]⁺, such as [Ir(dF(CF₃)ppy)₂(dtbpy)]Cl.

Photocatalysts (including transition metal photocatalysts) typically comprise one or more counterions. The counterion may be any counterion that is suitable for stabilizing the photocatalyst.

Typically, the counterion is negatively charges. That is, typically the counterion is an anion. Typical examples of anions include inorganic anions such as halo, borate and phosphate.

Typical inorganic anions include chlorate (Cl⁻), tetrafluoroborate (BF₄)⁻ and hexafluorophosphate (PF₆)⁻.

Optionally, the transition metal photocatalyst may be a hydrate. That is, the transition metal catalyst may contain water (H₂O).

Preferably the photocatalyst is a homogenous photocatalyst. That is, the photocatalyst exists in the same phase as the reactants. Typically, the photocatalyst is soluble in an 80% aqueous solution, such as an 85% or 90% aqueous solution. Aqueous solutions are preferred for solubility of nucleic acids.

The aqueous solubility of the photocatalysts may be known, or it may be determined using standard techniques. The metal and ligand system can be selected to adjust the aqueous solubility of the system.

### Nucleophile

The radical initiator may be used together with a nucleophilic compound. The nucleophilic compound may participate in the radical reaction that is initiated by the radical initiator.

This nucleophilic compound typically contains a thiol, seleno, hydroxyl or amino functional group, and the nucleophilic compound may be referred to as a thiol compound, seleno compound, hydroxyl compound or amine compound accordingly.

The nucleophile compound may be a Michael donor.

Preferably, the nucleophile compound is a thiol compound and/or the disulfide form thereof, such as those described below. The seleno forms of these compounds may also be used as nucleophiles in the present case.

Where the nucleophilic compound contains a hydroxyl group, the compound may be an alcohol, such as an alkyl alcohol.

Where the nucleophilic compound contains an amino group, this may be a primary or secondary amino group. The compound may be an amine, such as an alkyl amine.

The nucleophilic compound is preferably a small organic compound, such as a compound having a molecular weight of not more than 200, such as not more than 100.

The nucleophilic compound is preferably not in salt form.

The nucleophile is preferably a liquid at room temperature, such as 20° C.

### Thiol and Disulfide Forms Thereof

The radical initiator may be provided together with a thiol compound and/or the disulfide form thereof.

The thiol compound contains at least one thiol functional group (—SH), and may contain one, two or three thiol groups. Typically, the thiol compound contains one thiol group (monothiol substituted).

The thiol compound may additionally contain one or more additional functional groups. For example, the thiol compound may contain a functional group selected from hydroxyl, amino, and carboxy.

The thiol compound may contain one or more hydroxyl groups. For example the thiol compound may contain one, two or three hydroxyl groups. Typically, the thiol compound contains one hydroxyl group (monohydroxyl substituted).

The thiol compound may contain one or more carboxyl (—COOH) groups, and/or one or more the alkyl esters of such carboxyl groups. For example the thiol compound may contain one, two or three carboxyl groups. For example the thiol compound may contain one, two or three alkyl ester groups. The ester may be an ester of a methyl or ethyl alcohol with the carboxyl group, such as a methyl or they ester acid.

The thiol compound may contain one or more amino groups. For example the thiol compound may contain one, two or three hydroxyl groups.

The thiol compound is soluble in the reaction solvent, which solvent may be an aqueous solvent, such as a mixture of water and acetonitrile. The thiol compound is preferably water soluble.

The thiol compound may be a hydrocarbon having one, two or three thiol groups, and optionally substituted with one or more additional functional groups, as described above, such as hydroxyl, amino, and carboxyl, including the alkyl esters of the carboxyl groups.

The thiol compound may be an alkyl thiol, which may be optionally substituted with one or more additional functional groups, as described above, such as hydroxyl, amino, and carboxyl, including the alkyl esters of the carboxyl groups.

The thiol compound may be an amino acid or a polypeptide containing an amino acid, where a side chain of an amino acid residue containing a thiol group. Thus, the thiol compound may be cysteine or a polypeptide containing a cysteine residue, such as glutathione.

The thiol compound may be selected from the group consisting of 2-mercaptoethanol, methyl 2-mercaptoacetate, 2-mercaptoacetic acid, cysteamine, cysteine, glutathione, 2,3-mercaptosuccinic acid the esters thereof, thiophenol, benzyl mercaptan, tri-isopropylsilane thiol, methane thiol, and hydrogen disulphide (HS₂).

In an alternative to these thiols, the nucleophile can the sulfur-containing compound carbon disulfide (CS₂).

The thiol may be 2-mercaptoethanol (mercaptoethanol). This thiol is advantageously not flammable, which is in contrast to the exemplary boron-containing compounds used by Liu et al.

The invention also allows for the use of selenol compounds, and the diselenide forms thereof. The selenol compounds may be the same as the thiol compounds described above, where one or more thiol groups is replaced with a selenol group.

Each of the compounds described above, may also be provided as their oxidized forms, where a sulfur atom in a thiol group may be mono-oxidised or di-oxidised.

### Reaction Mixture and Solvent

The methods of the present case may be undertaken in solution, and this may be an aqueous solution, optionally containing one or more organic solvents.

The method may be performed in a solvent, such as an aqueous solvent. The aqueous solvent may be a mixture of water and one or more organic solvents that are miscible with water.

In one embodiment, the aqueous solvent includes acetonitrile.

The aqueous solvent system may be an acidic solvent system. The mixture may have a pH in the range pH 3 to less than pH 7, such as pH 4 to less than pH 7, such as pH 4 to pH 6, such as pH 4 to pH 5.

In the present case, a preferred solvent system for use is a water and acetonitrile mixture at about pH 4.5 and about 5.9.

A buffer may be provided to maintain the pH at a desired level. The buffer may be an acetate or phosphate buffer. The buffer is provided at an appropriate level, as will be clear to a skilled person.

A nucleoside or polynucleotide may be provided in a reaction solvent at an appropriate amount and concentration. These may be present at, for example 1 nM to 1 M.

A nucleoside may be present at a concentration in the range 1 μM to 1,000 mM, such as 0.1 mM to 100 mM, such as 1 mM to 100 mM.

A polynucleotide may be present at a concentration in the range 1 nM to 100 mM, such as 100 nM to 1 mM, such as 1 μM to 100 μM.

Each of the radical initiator and the nucleophile are used at appropriate amounts and concentrations.

The radical initiator may be present at a concentration in the range 1 μM to 100 mM, such as 10 μM to 10 mM.

The nucleophile may be present at a concentration in the range 1 mM to 5 M, such as 10 mM to 2 M, such as 100 mM to 1.5 M

The methods may be performed at ambient (or room) temperature. For example, the reaction may be performed at a temperature in the range 10 to 25° C.

If necessary, the reaction may be performed at a lower temperature, such as in the range 0 to 10° C., or at higher temperature, such as in the range 25 to 80° C.

Where the radical initiator is photo-initiated the methods of the present case will include irradiation of a reaction mixture with light of an appropriate wavelength. This light may be incident onto the mixture continuously through the reaction, initially only, or in pulses throughout the reaction, as needed.

Similarly, where the radical initiator is thermally-initiated the methods of the present case will include heating of a reaction mixture to an appropriate temperature. This heating may be continuous through the reaction, initially only, or in pulses throughout the reaction, as needed.

A nucleoside or a polynucleotide, such as present within a sample nucleotide sequence, may be treated with a radical initiator, optionally with the nucleophile, for sufficient time to allow for conversion of 5mC to DHT and/or 5caC to DHU.

The progress of a conversion reaction may be judged analytically, for example by monitoring the consumption of the starting material nucleoside or polynucleotide and/or monitoring the formation of a reaction product. The reaction may be halted when substantially all of the staring material is consumed, and/or the formation of the product is considered to have a reached a contact maximum. Analytical techniques suitable for reaction monitoring in the present case include UV-vis spectroscopy, LC-MS and NMR spectroscopy.

The reaction for a treatment of a modified cytosine with a radical initiator, optionally with the nucleophile, may be at most 24 hours, such as at most 18 hours, such as at most 12 hours, such as at most 6 hours, such as at most 2 hours, such as at most 1 hour.

The reaction for a treatment of a modified cytosine with a radical initiator, optionally with the nucleophile, may be at least 5 minutes, such as at least 10 minutes, such as at least 30.

The inventors have found that polynucleotides require a shorter reaction time compared with a simple nucleoside.

The reaction times may be reduced by, for example, increasing the nucleophile concentration, increasing the radical initiator concentration, and decreasing the nucleoside or polynucleotide concentration.

After treatment, the treated nucleoside or polynucleotide may be at least partially purified. Here, the polynucleotide may be separate from the radical initiator and the nucleophile, where present. Techniques for the work-up and isolation of nucleoside and polynucleotides are well known in the art.

Where a method of the invention includes a step for the generation of DHT from 5mC or the generation of DHU from 5caC, that step may be performed in one-pot. Thus, the reaction is undertaken without the isolation or purification of any intermediate forms. Here, pot may broadly refer to a reaction flask, a vial or a well in a well plate, as commonly used in the field of nucleoside preparation and polynucleotide amplification and sequencing.

### Kits

In a further aspect the present invention provides a kit comprising:
(a) a radical initiator as described herein;
(b) a nucleophile as described herein;
(c) optionally a solvent.

The kit may be provided in a suitable container and/or with suitable packaging.

Optionally, the kit may include instructions for use, e.g., written instructions on how to use the kit in a method of detecting 5mC in a nucleotide sample.

A kit may further comprise a population of control polynucleotides comprising one or more modified cytosine residues, for example cytosine (C), 5-methylcytosine (5mC), 5-hydroxymethylcytosine (5hmC) or 5-formylcytosine (5fC). In some embodiments, the population of control polynucleotides may be divided into one or more portions, each portion comprising a different modified cytosine residue.

The kit may include instructions for use in a method of identifying a modified cytosine residue or a nucleotide residue as described above.

A kit may include one or more other reagents required for the method, such as buffer solutions, sequencing and other reagents. A kit for use in identifying modified cytosines may include one or more articles and/or reagents for performance of the method, such as means for providing the test sample itself, including DNA and/or RNA isolation and purification reagents, and sample handling containers (such components generally being sterile).

A kit may include sequencing adapters and one or more reagents for the attachment of sequencing adapters to the ends of isolated nucleic acids, such as T4 ligase.

A kit may include one or more reagents for the amplification of a population of nucleic acids using the amplification primers. Suitable reagents may include a thermostable polymerase, for example a high discrimination polymerase, dNTPs and an appropriate buffer.

### Methods

The methods of the invention may be used to detect a 5mC or a 5caC residue in sample nucleotide sequence.

Thus, the invention provides a method for modifying a polynucleotide, the method comprising converting a 5-methylcytosine (5mC) residue in a polynucleotide directly to a dihydrothymine (DHT) residue.

In this method, the 5mC residue is reduced and deaminated. The reduction is the reduction of the C5-C6 bond in the 5mC residue, and the deamination is the loss of the amino group at the C4 position, which is replaced with hydroxyl. As noted previously, the hydroxyl group tautomerises to give the preferred keto form, as observed in the DHT residue.

This method for preparing a DHT residue may be incorporated into a method for detecting a modified cytosine residue within a sample nucleotide sequence. Thus, the invention provides a method of identifying a modified cytosine residue in a sample nucleotide sequence, the method comprising;
(i) providing a population of polynucleotides which comprise the sample nucleotide sequence,
(ii) reducing and deaminating a 5-methylcytosine (5mC) residue within the population,
(iii) sequencing the polynucleotides in the population or derivatives thereof following step (ii) to produce a treated nucleotide sequence, and;
(iv) identifying the residue in the treated nucleotide sequence which corresponds to a modified cytosine residue in the sample nucleotide sequence.

The methods of the invention are also suitable for converting a 5caC residue to the corresponding DHU form. The methods of the invention therefore provide alternative reaction conditions for this conversion over the methods described in the prior art.

It follows then that the methods of the invention may be used in conventional sequencing methods where the production of a 5caC residue is a step in the sequencing methodology, or more generally where the sequencing methodology looks to detect the presence of a 5caC residue within a polynucleotide. These methods may include methods for detecting a modified cytosine residue, such as 5mC, within a sample nucleotide sequence, where the method includes the preparation of 5caC.

If follows then, that the invention also provides a method of identifying a modified cytosine residue in a sample nucleotide sequence, the method comprising:
(i) providing a population of polynucleotides which comprise the sample nucleotide sequence,
(ii) oxidising a first portion of said population,
(iii) treating the oxidised first portion of said population with a radical initiator optionally together with a nucleophile,
(iv) sequencing the polynucleotides in the first portions of the population or derivatives thereof following steps ii) and iii) to produce a first nucleotide sequence, and;
(v) identifying the residue in the first nucleotide sequences which corresponds to a modified cytosine residue in the sample nucleotide sequence.

Within step (ii), the oxidation may be the oxidation of a C5 methyl group in a modified cytosine residue, for example the oxidation the C5 methyl group of 5mC. The product of this reaction may include 5caC. Thus, 5mC may be converted to 5caC in this step.

The oxidation step may also include the oxidation of a C5 hydroxymethyl group in a modified cytosine residue. The product of this reaction may include 5caC. Thus, 5hmC may be converted to 5caC in this step.

Methods for performing an oxidation of a polynucleotide, such as to give 5caC, are known in the art. The conversion of 5mC or 5hmC to 5caC may be an oxidation by an enzyme, such as oxidation by an oxidase. Preferably, the oxidation is an oxidation by a ten-eleven-translocation (TET) oxygenase, such as an oxygenase selected from TET1, TET2 and TET3.

The invention also provides a method of identifying a nucleotide in a sample nucleotide sequence, the method comprising:
(i) providing a population of polynucleotides which comprise the sample nucleotide sequence,
(ii) treating the population with a radical initiator optionally together with a nucleophile to produce treated polynucleotides,
(iii) sequencing the treated polynucleotides following (ii) or derivatives thereof, to obtain a nucleotide sequence comprising a transformed nucleotide corresponding to the nucleotide in the sample nucleotide sequence, and;
(iv) identifying the transformed nucleotide in the nucleotide sequence, thereby identifying the nucleotide in the sample nucleotide sequence.

The transformed nucleotide in the nucleotide sequence may comprise a thymine residue. Here the nucleotide in the sample nucleotide sequence is identified as thymine in the sequenced nucleotide sequence.

The nucleotide identified in the sample nucleotide sequence may comprise an adenine residue. In these cases, the adenine residue in the sample nucleotide sequence is detected as the transformed nucleotide in step (iv). In some cases where the transformed nucleotide comprises a thymine residue, the nucleotide is identified as an A-to-T transition.

The nucleotide identified in the sample nucleotide sequence may comprise a guanine residue. In these cases, the guanine residue in the sample nucleotide sequence is detected as the transformed nucleotide in step (iv). In some of these cases where the transformed nucleotide comprises a thymine residue, the nucleotide is identified as a G-to-T transition.

The nucleotide identified in the sample nucleotide sequence may comprise a cytosine residue. In these cases, the cytosine residue in the sample nucleotide sequence is detected as the transformed nucleotide in step (iv). In some of these cases where the transformed nucleotide comprises a thymine residue, the nucleotide is identified as a C-to-T transition.

The nucleotide identified in the sample nucleotide sequence may comprise a modified cytosine residue, such as 5caC or 5mC. In these cases, the modified cytosine residue in the sample nucleotide sequence is detected as the transformed nucleotide in step (iv). In some of these cases where the transformed nucleotide comprises a thymine residue, the nucleotide is identified as a C-to-T transition.

The methods of the invention are suitable for use in the analysis of a sample nucleotide sequence. This sample contains a polynucleotide, such as a polynucleotide population, and it may contain a mixture of polynucleotides.

Any sample nucleotide sequence may be a copied sample. For example, the sample nucleotide sequence, the population of polynucleotides which comprises the sample nucleotide sequence, or a portion of the population, may be copied before sequencing. Copying of a sample may include generation of a complementary nucleotide sequence, such as the generation of a double-stranded polynucleotide by enzymatic polymerisation or by primer extension. Copying of a sample may include amplification of the nucleotide sequence, such as by polymerase chain reaction. Copying may be carried out following a step of treatment with the radical initiator, optionally together with the nucleophile. In some cases, copying may be carried out prior to treatment with a radical initiator, optionally together with the nucleophile.

Any sample nucleotide sequence may be an amplified sample. One or more populations may be made of the sample, and each population may be subjected to a different sequencing and identification process. Thus, the methods of the invention may be used in relation to one population to identify a modified cytosine residue in the sample nucleotide sequence, for example to identify 5mC and/or 5caC. The other populations may be used within methods to determine the presence of alternative modified residues, such as alternatively modified cytosine residues.

The sample nucleotide sequence, the population of polynucleotides which comprises the sample nucleotide sequence, or a portion of the population may be amplified before sequencing. Amplification may be carried out before treatment with the radical initiator, which may be followed by sequencing to identify the sequence of the sample nucleotide. Amplification may be carried out after treatment with the radical initiator and the amplified sample may be sequenced according to step (iii). The transformed nucleotide may then be identified as a base transition when comparing sequencing results obtained in this way.

In the methods of the invention, a modified polynucleotide is prepared—by converting 5mC to DHT and/or 5caC to DHU—and the sequence of the modified polynucleotide is determined.

The methods of the invention allow for this modified polynucleotide to be compared against a polynucleotide sequence that is not treated. A comparison between these sequences can show where there has been a C to T change upon treatment. Thus, the presence of 5mC and/or 5caC may be determined.

Thus, a sample nucleotide sequence may include an untreated portion and a treated portion. The polynucleotides in each portion may be sequenced, and compared against each other to allow for identification of a modification in the treated portion.

In the methods of the present case, any step of identifying a modified cytosine in a sample includes the step of treating a population of a nucleotide sample, such that 5mC and/or 5caC residues within a polynucleotide are converted to DHT and DHU residues respectively. The treated polynucleotide may be sequenced and the residue in the treated nucleotide sequence which corresponds to a modified cytosine residue in the sample nucleotide sequence may be identified. Here, identification follows a change in sequenced residues between the sample and the treated polynucleotides. Thus, 5mC and 5caC, which are read as C are read as T in the treated sequence. Thus, the presence of a thymine residue in the treated nucleotide sequence is indicative that the modified cytosine residue in the sample nucleotide sequence is 5mC or 5caC.

Thus, in one embodiment of the invention, a sample nucleotide sequence may be made into two or three populations. A first population may be analysed using the methods of the invention. Thus, a 5mC residue in a polynucleotide may be converted directly to DHT or indirectly to DHU, via a 5caC residue. Here, a 5hmC residue may be converted to DHU, via a 5caC residue. The resulting polynucleotide may then be sequenced and the modified cytosine residue identified in the usual way. This method may be combined with the methods described below for the second and/or third populations.

A second population may be treated with a protecting agent, to protect a 5hmC residue in a polynucleotide, for example as glucose-protected 5-hydroxymethylcytosine (5gmC). The treated population may then be subsequently further treated to convert a 5mC residue in a polynucleotide to a 5caC residue, and then this 5caC residue to a DHU residue. The resulting polynucleotide may then be sequenced and the modified cytosine residue identified in the usual way.

A third population may be treated with an oxidising agent to convert a 5hmC residue in a polynucleotide to a 5fC residue, for example with a Ru-based oxidizing agent. In a subsequent step, the 5fC may be converted to a DHU residue. The resulting polynucleotide may then be sequenced and the modified cytosine residue identified in the usual way.

A fourth population may be treated with a reducing agent to convert 5fC residue in polynucleotide to a 5hmC residue. In a subsequent step, the polynucleotide may be treated with a protecting agent, to protect a 5hmC residue in a polynucleotide, for example as glucose-protected 5-hydroxymethylcytosine (5gmC). The resulting polynucleotide may then be sequenced and the modified cytosine residue identified in the usual way.

Methods for the preparation of 5fC from 5hmC are described by one of the present inventors in WO 2013/017853, the contents of which are hereby incorporated by reference herein. Example oxidising agents described here, and suitable for use in the present case, are perruthenate oxidising agents, such as KRuO₄.

An analysis of a sample nucleotide sequence with multiple populations is described, for example, by Uu et al. and WO 2019/136413. The methods for transforming 5mC, 5hmC and 5fC, and the accompanying methods of analysis, disclosed in these documents are incorporated by reference herein.

The sample nucleotide sequence may be a genomic sequence. For example, the sequence may comprise all or part of the sequence of a gene, including exons, introns or upstream or downstream regulatory elements, or the sequence may comprise genomic sequence that is not associated with a gene. In some embodiments, the sample nucleotide sequence may comprise one or more CpG islands.

Suitable polynucleotides include DNA, preferably genomic DNA, and/or RNA, such as genomic RNA (e.g. mammalian, plant or viral genomic RNA), mRNA, tRNA, rRNA and non-coding RNA.

The polynucleotides comprising the sample nucleotide sequence may be obtained or isolated from a sample of cells, for example, mammalian cells, preferably human cells.

Suitable samples include isolated cells and tissue samples, such as biopsies, as well as blood samples. Cell sample may be derived a range of cell types including embryonic stem cells, neural cells, etc.

Suitable cells include somatic and germ-line cells.

Suitable cells may be at any stage of development, including fully or partially differentiated cells or non-differentiated or pluripotent cells, including stem cells, such as adult or somatic stem cells, foetal stem cells or embryonic stem cells.

Suitable cells also include induced pluripotent stem cells (iPSCs), which may be derived from any type of somatic cell in accordance with standard techniques.

For example, polynucleotides comprising the sample nucleotide sequence may be obtained or isolated from neural cells, including neurons and glial cells, contractile muscle cells, smooth muscle cells, liver cells, hormone synthesising cells, sebaceous cells, pancreatic islet cells, adrenal cortex cells, fibroblasts, keratinocytes, endothelial and urothelial cells, osteocytes, and chondrocytes.

Suitable cells include disease-associated cells, for example cancer cells, such as carcinoma, sarcoma, lymphoma, blastoma or germ line tumour cells.

Suitable cells include cells with the genotype of a genetic disorder such as Huntington's disease, cystic fibrosis, sickle cell disease, phenylketonuria, Down syndrome or Marfan syndrome.

Methods of extracting and isolating genomic DNA and RNA from samples of cells are well-known in the art. For example, genomic DNA or RNA may be isolated using any convenient isolation technique, such as phenol/chloroform extraction and alcohol precipitation, caesium chloride density gradient centrifugation, solid-phase anion-exchange chromatography and silica gel-based techniques.

In some embodiments, whole genomic DNA and/or RNA isolated from cells may be used directly as a population of polynucleotides as described herein after isolation. In other embodiments, the isolated genomic DNA and/or RNA may be subjected to further preparation steps.

A sample may also be a blood sample, from which circulating free DNA (cfDNA) or circulating tumour DNA (ctDNA) may be extracted.

The genomic DNA and/or RNA may be fragmented, for example by sonication, shearing or endonuclease digestion, to produce genomic DNA fragments. A fraction of the genomic DNA and/or RNA may be used as described herein. Suitable fractions of genomic DNA and/or RNA may be based on size or other criteria. In some embodiments, a fraction of genomic DNA and/or RNA fragments which is enriched for CpG islands (CGIs) may be used as described herein.

The genomic DNA and/or RNA may be denatured, for example by heating or treatment with a denaturing agent. Suitable methods for the denaturation of genomic DNA and RNA are well known in the art.

In some embodiments, the genomic DNA and/or RNA may be adapted for sequencing before treatment, for example before treatment to reduce and deaminate a modified cytosine, such as before treatment to reduce, deaminate and decarboxylate a modified cytosine. The nature of the adaptations depends on the sequencing method that is to be employed. For example, for some sequencing methods, primers may be ligated to the free ends of the genomic DNA and/or RNA fragments following fragmentation. In other embodiments, the genomic DNA and/or RNA may be adapted for sequencing after treatment, as described herein.

Following fractionation, denaturation, adaptation and/or other preparation steps, the genomic DNA and/or RNA may be purified by any convenient technique.

Following preparation, the population of polynucleotides may be provided in a suitable form for further treatment as described herein. For example, the population of polynucleotides may be in aqueous solution in the absence of buffers before treatment as described herein.

Polynucleotides for use as described herein may be single or double-stranded.

Preferably, double-stranded polynucleotides for use as described herein are denatured into single-stranded polynucleotides prior to treatment. For example, double-stranded polynucleotides may be adapted for sequencing, followed by denaturation such as by heating or under alkaline condition to provide single-stranded polynucleotides, and then treated as described herein. Polynucleotides may then be amplified after treatment, or primer extension may be carried out on single-stranded polynucleotides, to generate double-stranded polynucleotides for library preparation and sequencing.

The population of polynucleotides may be divided into two, three, four or more separate portions, each of which contains polynucleotides comprising the sample nucleotide sequence. These portions may be independently treated and sequenced, such as described herein.

Preferably, the portions of polynucleotides are not treated to add labels or substituent groups to 5caC residues in a sample nucleotide sequence before treatment, for example before treatment to reduce, deaminate and decarboxylate this modified cytosine.

As described above, polynucleotides may be adapted after treatment to be compatible with a sequencing technique or platform. The nature of the adaptation will depend on the sequencing technique or platform. For example, for Solexa-Illumina sequencing, the treated polynucleotides may be fragmented, for example by sonication or restriction endonuclease treatment, the free ends of the polynucleotides repaired as required, and primers ligated onto the ends.

Polynucleotides may be sequenced using any convenient low or high throughput sequencing technique or platform, including Sanger sequencing (43), Solexa-Illumina sequencing (44), Ligation-based sequencing (SOLiD™) (45), pyrosequencing (46); strobe sequencing (SMRT™) (47, 48); semiconductor array sequencing (Ion Torren™) (49); and nanopore sequencing (ION).

Suitable protocols, reagents and apparatus for polynucleotide sequencing are well known in the art and are available commercially.

The residues at positions in the first and other sequences which correspond to cytosine in the sample nucleotide sequence may be identified.

The modification of a cytosine residue at a position in the sample nucleotide sequence may be determined from the identity of the residues at the corresponding positions in the first, second and, optionally, third nucleotide sequences, as described above. As noted previously, the methods of the invention effectively enable a C to T transition between the sample nucleotide sequence and the treated sequences.

The extent or amount of cytosine modification in the sample nucleotide sequence may be determined. For example, the proportion or amount of 5mC or 5caC in the sample nucleotide sequence compared to unmodified cytosine may be determined.

Polynucleotides as described herein, for example the population of polynucleotides or 1, 2, 3, or all 4 of the first, second, third and fourth portions of the population, may be immobilised on a solid support.

A solid support is an insoluble, non-gelatinous body which presents a surface on which the polynucleotides can be immobilised.

Examples of suitable supports include glass slides, microwells, membranes, or microbeads. The support may be in particulate or solid form, including for example a plate, a test tube, bead, a ball, filter, fabric, polymer or a membrane. Polynucleotides may, for example, be fixed to an inert polymer, a 96-well plate, other device, apparatus or material which is used in a nucleic acid sequencing or other investigative context. The immobilisation of polynucleotides to the surface of solid supports is well-known in the art. In some embodiments, the solid support itself may be immobilised. For example, microbeads may be immobilised on a second solid surface.

In some embodiments, the first, second, third and/or fourth portions of the population of polynucleotides may be amplified before sequencing. Preferably, the portions of polynucleotide are amplified following the treatment with bisulfite.

Suitable methods for the amplification of polynucleotides are well known in the art.

Following amplification, the amplified portions of the population of polynucleotides may be sequenced.

Nucleotide sequences may be compared and the residues at positions in the first, second and/or third nucleotide sequences which correspond to cytosine in the sample nucleotide sequence may be identified, using computer-based sequence analysis.

Nucleotide sequences, such as CpG islands, with cytosine modification greater than a threshold value may be identified. For example, one or more nucleotide sequences in which greater than 1%, greater than 2%, greater than 3%, greater than 4% or greater than 5% of cytosines are 5-methylated and/or 5-carboxylated may be identified.

Computer-based sequence analysis may be performed using any convenient computer system and software. A typical computer system comprises a central processing unit (CPU), input means, output means and data storage means (such as RAM). A monitor or other image display is preferably provided. The computer system may be operably linked to a DNA and/or RNA sequencer.

The methods of the invention also have applicability to the reaction of the 5mC and 5caC nucleosides. Using the methods described herein, a further aspect of the invention provides methods where 5mC is converted to the nucleoside DHT, and 5caC is converted to the nucleoside DHU.

### Screening Methods

The present inventors have established for the first time that 5mC may be directly converted to DHT in a one step, and one pot process. In the worked examples described herein this change is effected by a radical initiator in the presence of a thiol compound, and specifically through the use of Ir[dF(CF₃)ppy]₂(dtbpy)Cl together with mercaptoethanol.

Now that the direct conversion of 5mC to DHT is demonstrated, the inventors understand that alternative reagents and conditions may be identified that also effect this conversion. Thus, the present invention therefore also provides a method for identifying reaction conditions for the formation of DHT from 5mC.

Thus, there is provided a method for identifying a reaction condition for the transformation of a 5-methylcytosine (5mC) to a dihydrothymine (DHT), the method comprising the steps of:
(i) treating a 5-methylcytosine (5mC) with one or more test reagents;
(ii) detecting the presence of dihydrothymine (DHT) as a product of the treatment

The treatment is performed in one-pot.

The 5mC may be a nucleoside or a residue in a polynucleotide.

The method may also include the step of treating a 5caC with the one or more test reagents, and subsequently detecting the presence of dihydrouracil (DHU) as a product of the treatment. The 5caC may be a nucleoside or a residue in a polynucleotide.

The presence of DHT in a reaction product may be determined using standard and appropriate analytical techniques, such as those described herein. Thus, LC-MS and NMR may be used to analyse the reaction product. The consumption of the 5mC may also be monitored and analysed, using LC-MS, for example.

### Salts, Solvates and Other Forms

A reference to a compound described herein, is also a reference to a salt of that compound.

These salts include all salts, such as, without limitation, acid addition salts of strong mineral acids such as HCl and HBr salts and addition salts of strong organic acids such as a methanesulfonic acid salt. Further examples of salts include sulfates and acetates such as trifluoroacetate or trichloroacetate.

A reference to a compound described herein, is also a reference to a solvate of that compound. Examples of solvates include hydrates

A compound described herein, includes a compound where an atom is replaced by a naturally occurring or non-naturally occurring isotope. In one embodiment the isotope is a stable isotope. Thus a compound described herein includes, for example deuterium containing compounds and the like. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Any of the compound described herein, may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₆alkyl includes n-propyl and iso-propyl; butyl includes n-, iso-, sec-, and tert-butyl; methoxyphenyl includes ortho-, meta-, and para-methoxyphenyl).

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallization and chromatographic means) of such isomeric forms are either known in the art or are readily obtained by adapting the methods taught herein, or known methods, in a known manner.

One aspect of the present invention pertains to compounds in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to an equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

### Other Preferences

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

### Experimental and Results

The methods of the invention are exemplified in a series of six experiments.

In the first, a 5caC nucleoside (2′-deoxy-5-carboxycytidine) is treated with a radical initiator and a thiol to give a DHU nucleoside product. In the second, a 5caC-containing 10-mer oligomer (a nucleotide sequence) is treated with a radical initiator and a thiol to give a DHU-containing oligomer product. In the third, a 5mC nucleoside (2′-deoxy-5-methylcytidine) is treated with a radical initiator and a thiol to give a DHT nucleoside product.

In all three experiments above, the radical initiator is [Ir(dF(CF₃)ppy)₂(dtbpy)]Cl and the thiol is mercaptoethanol. The reactions were performed at room temperature (20° C.) in a solution of water and acetonitrile, buffered to pH 4.5. The reaction mixture was irradiated at 450 nm throughout the reaction. The reaction products were analysed by LC-MS and ¹H NMR, amongst others.

The reaction conditions are described in greater detail below, together with the details of the three additional experiments.

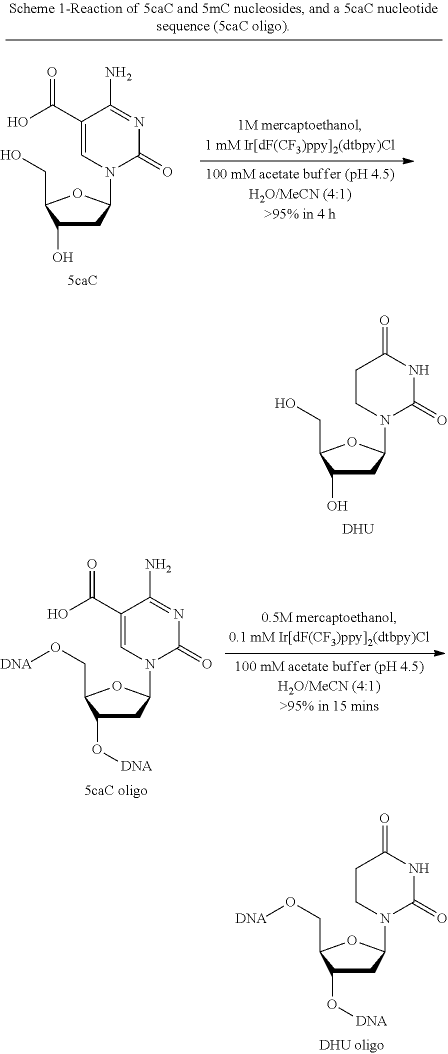

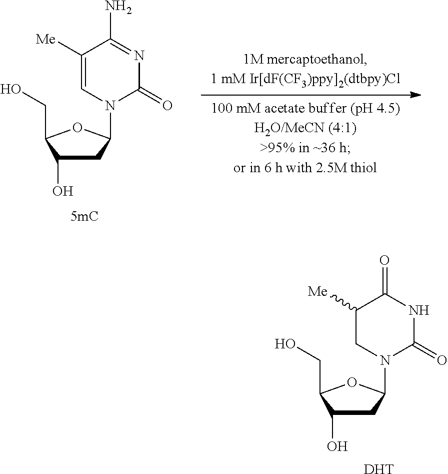

The sequencing methods of the invention are exemplified in three further experiments.

In the first, the identification of base-pairing conversion via next-generation sequencing is shown using synthetic oligos. In the second, a two-step 5mC sequencing method using lambda-phage DNA as a model is demonstrated. In the third, the conditions used during the two-step 5mC sequencing method is optimised.

### General Experimental and Analytical Conditions

2′-Deoxy-5-methylcytidine and 2′-deoxycytidine are available from Fisher. 2′-Deoxy-5-carboxycytidine is available from Berry & Associates. Dihydro-2′-deoxyuridine is available from Insight Biotechnology Ltd. Ir[dF(CF₃)ppy]₂(dtbpy)PFe is available from Sigma Aldrich or Manchester Organics.

All solvents, buffers and reagents were prepared by standard procedures or used as supplied from commercial sources Sigma-Aldrich, Alfa Aesar, or Fisher Scientific, and all reactions were performed at room temperature (20° C.) unless otherwise stated.

Mercaptoethanol refers to 2-mercaptoethanol (β-mercaptoethanol).

Ir[dF(CF₃)ppy]₂(dtbpy)Cl was prepared from the PFe salt as follows:

Dowex ion exchange resin (1×8 chloride mesh) was washed with 0.5 M NaCl (aq.) (3×20 mL) and water (20 mL). Ir[dF(CF₃)ppy]₂(dtbpy)PFe (100 mg) was dissolved in MeCN/H₂O (20 mL, 1:1 by volume), and this solution was filtered five times through a column containing the prepared Dowex resin. The column was washed with water (10 mL) and the solvent removed from the combined filtrate by lyophilisation.

Oligonucleotides were synthesised by ATDBio (phosphoramidite synthesis with HPLC purification) and used as supplied by the manufacturer.

Liquid chromatography-mass spectrometry (LC-MS) analysis for nucleoside samples was carried out on a Bruker amaZon X Ion Trap MS using a Supelcosil LC-18-S nucleoside column (Sigma-Aldrich, 4.6 mm, 5 μm; 0.5 to 25% MBCN in water, with 0.1% formic acid).

Liquid chromatography-mass spectrometry (LC-MS) analysis for oligomer samples was carried out on a Bruker amaZon X Ion Trap MS using an Acquity Premier BEH C18 column (Waters, 2.1 mm, 1.7 μm; 5 to 40% MeOH in water buffered with 10 mM TEA, 100 mM HFIP). The column was heated to 60° C. for analysis of annealed oligomer reactions.

Proton and carbon nuclear magnetic resonance (1H NMR and 13C NMR) spectra were acquired with a Bruker 500 MHz DCH Cryoprobe Spectrometer, using deuterated solvents as indicated. Chemical shifts (6) are reported in parts per million (ppm) relative to the residual solvent, and coupling constants (J) are reported in hertz (Hz). Multiplicity is reported using combinations of the following abbreviations: s=singlet, d=doublet, t=triplet, m=multiplet/overlapping peaks, br=broad. Analysis of NMR spectra was performed using MestReNova software.

High resolution mass spectra (HRMS) for nucleosides were recorded on a ThermoFinnigan Orbitrap Classic mass spectrometer.

### Nucleoside Reaction Conditions

To a 1.5 mL vial were added the following components:
264.8 μL water,
50 μL of 1 M, pH 4.5 aqueous sodium acetate buffer (final reaction concentration 100 mM),
100 μL Ir(dF-CF₃-ppy)₂(dtbpy)₃Cl photocatalyst, 5 mM stock solution in MBCN (final reaction concentrations 1 mM photocatalyst/20% MeCN),
35.2 μL neat mercaptoethanol (final reaction concentration 1 M),
50 μL nucleoside, 100 mM stock solution in water (final reaction concentration 10 mM). The solution was stirred under an air atmosphere with continual illumination by blue LEDs (450 nm) in a PhotoRedOx Box (HepatoChem). Reaction samples were collected at desired timepoints, diluted three times with water and frozen until analysis by LC-MS.

The reaction was performed at room temperature (20° C.).

The reaction was typically deemed complete—as judged by consumption of the starting material nucleoside (LC-MS and UV-vis), and the maximum production of desired product (LC-MS and UV-vis).

### Reaction of 5caC Nucleoside

The 5caC nucleoside was reacted as described in the nucleoside reaction conditions described above. The reaction product was identified as DHU.

The reaction with 5caC was typically deemed complete—as judged by consumption of the starting material nucleoside (LC-MS and UV-vis), and the maximum production of desired product (LC-MS and UV-vis)—after 5.5 hours. The half-life of 5caC under the standard conditions is −90 minutes. Under these conditions the yield of DHU was adjudged to be >95% after 4 hours.

It was found that the reaction time could be reduced when the thiol concentration was increased, or the nucleoside concentration was reduced. A reduction in the amount of the photocatalyst is associated with an increase in reaction timing.

Retention time on LCMS: 2.8 mins (starting material elutes at 3.8 mins). Masses [M+H]+ 115.14, 231.08, 461.10 (nucleobase, nucleoside and dimer).

¹H NMR (500 MHz, D₂O): δ 6.15 (ddd, J=8.1, 5.0, 1.8 Hz, 1H), 4.26 (ddt, J=7.5, 5.5, 2.7 Hz, 1H), 3.80 (dtd, J=5.7, 3.9, 1.8 Hz, 1H), 3.70-3.62 (m, 1H), 3.60-3.53 (m, 1H), 3.48-3.36 (m, 2H), 2.64 (dddd, J=8.6, 6.8, 5.1, 1.9 Hz, 2H), 2.25-2.15 (m, 1H), 2.02 (dddd, J=14.2, 8.2, 3.8, 1.9 Hz, 1H). ¹³C NMR (500 MHz, D₂O): 173.85, 154.18, 85.10, 83.97, 70.77, 61.50, 35.65, 35.13, 30.00. HRMS [M−H]⁻ for [C₉H₁₃N₂O₅]⁻ calculated 229.0824, found 229.0834.

The NMR data was in agreement with a commercial standard used as a reference.

### Reaction of 5mC Nucleoside

The 5mC nucleoside was reacted as described in the nucleoside reaction conditions described above. The reaction product was identified as DHT.

The reaction with 5mC was typically deemed complete—as judged by consumption of the starting material nucleoside (LC-MS and UV-vis), and the maximum production of desired product (LC-MS and UV-vis)—after 36 hours. Under these conditions the yield of DHT was adjudged to be >95% after 36 hours.

In a further experiment, the thiol concentration in the reaction mixture was increased to 2.5 M. Here, the reaction was deemed completed after 10 hours. Under these conditions the yield of DHT was adjudged to be >95% after 8 hours, and after 6 hours also.

Retention time on LCMS: 4.6 mins and 5.0 mins (starting material elutes at 4.4 mins). Masses [M+H]+ 129.18, 245.15, 489.14 (nucleobase, nucleoside and dimer).

¹H NMR (500 MHz, D₂O): δ 6.21-6.12 (m, 1H), 4.26 (dt, J=6.8, 3.8 Hz, 1H), 3.78 (ddt, J=7.8, 5.9, 3.9 Hz, 1H), 3.69-3.38 (m, 3H), 3.19-3.11 (m, 1H), 2.72 (ddd, J=9.5, 7.4, 5.6 Hz, 1H), 2.26-2.13 (m, 1H), 2.03 (ddt, J=13.0, 6.4, 3.3 Hz, 1H), 1.10 (dd, J=7.0, 3.5 Hz, 3H) (complex multiplets due to two diastereomers). ¹³C NMR (126 MHz, D₂O) δ 176.74, 176.50, 154.33, 154.08, 85.08, 83.88, 83.75, 70.78, 70.40, 61.55, 61.45, 41.89, 35.28, 35.02, 34.53 (extra peaks due to two diastereomers). HRMS [M+H]⁺ for [C₁₀H₁₇N₂O₅]⁺ calculated 245.1137, found 245.1133.

### Reaction of a, C, T and G Nucleosides

Unmodified A, C, T and G nucleosides were individually treated under the nucleoside reaction conditions described above. The mixture of nucleoside and reagents was monitored by LC-MS. Essentially no change in any of the nucleosides was observed over 10 hours (the relative amount was constant throughout the treatment period, and no reaction product peaks were observed.
A—Retention time on LCMS: 5.1 mins
C—Retention time on LCMS: 3.4 mins
T—Retention time on LCMS: 5.6 mins
G—Retention time on LCMS: 5.2 mins

In an initial study, no significant reaction of 5hmC and 5fC was observed under the nucleoside reaction conditions.

### Oligomer Reaction Conditions

To a 1.5 mL vial were added the following components:
50 uL of 1 M, pH 4.5 aqueous sodium acetate buffer (final reaction concentration 100 mM),
100 uL photocatalyst, 0.5 mM stock solution in MeCN (final reaction concentrations 0.1 mM photocatalyst/20% MeCN),
17.6 uL neat mercaptoethanol (final reaction concentration 0.5 M),
50-100 uL oligonucleotide aqueous stock solution (dependent on the concentration as supplied by the manufacturer; final reaction concentration 25 μM),
The volume was then made up to total 500 uM with water.

The solution was stirred under an air atmosphere with continual illumination by blue LEDs (450 nm) in a PhotoRedOx Box (HepatoChem). Reaction samples were collected every 15 minutes, purified using mini Quick Spin Oligo columns (Roche) according to manufacturers protocols, and diluted twice with water.

The reaction was typically deemed complete—as judged by consumption of the starting material nucleoside (LC-MS), and the maximum production of desired product (LC-MS)—after 30 minutes.

Reaction of 5caC-Containing 10-Mer Oligomer 1

Oligomer 1 (SEQ ID NO:1) was reacted as described in the oligomer reaction conditions as described above.
Oligomer 1: 5′-CTTAC[5caC]CTGA-3′, single strand, molecular weight 3007
Retention time: 4.7 mins
m/z 1502.42 (2—ion)
After 15 mins incubation:
Retention time: 4.5 mins
m/z 1481.61 (2—ion); predicted molecular weight for DHU oligo: 2965.98

As judged by LC-MS, the conversion of the oligomer to the DHU form was >98% after 15 minutes.

Reaction of 5caC-Containing 10-Mer Oligomer 2

Oligomer 2 (SEQ ID NO:2) was reacted as described in the oligomer reaction conditions as described above.
Oligomer 2: 5′-TCAG[5caC]GTAAG-3′, single strand, molecular weight 3096
Retention time: 4.8 mins
m/z 1547.01 (2—ion)
After 15 mins incubation:
Retention time: 4.6 mins
m/z 1526.96 (2—ion); predicted molecular weight for DHU oligo: 3054.99

From preliminary experiments, and as judged by LC-MS, the conversion of the oligomer to the DHU form was >70% after 15 minutes.

Reaction of 5caC-Containing 10-Mer Oligomer 3

Oligomer 1 (SEQ ID NO:1) was annealed with its complementary strand Oligomer 3 (SEQ ID NO:3): the two oligomers were mixed at 40 uM and 45 uM (respectively) in DNA annealing buffer (10 mM sodium phosphate, pH 7.0, with 0.2 M NaCl), heated to 95° C. and cooled to room temperature (2° C. per minute). The annealed mixture was reacted as described in the oligomer reaction conditions as described above.

### Zero-Hour Reaction Sample:

Oligomer 1 (forward strand): 5′-CTTAC[5caC]CTGA-3′, molecular weight 3096
   Retention time: 3.8 mins
   m/z 1502.48 (2—ion)
Oligomer 3 (reverse strand): 5′-TCAGGGTAAG-3′, molecular weight 3092
Retention time: 3.3 mins
m/z 1544.99 (2—ion)
After 15 mins incubation, a new UV absorbance appeared:
Retention time: 3.4 mins
m/z 1481.57 (2—ion); predicted molecular weight for DHU oligo: 2965.98

From preliminary experiments, and as judged by LC-MS, the conversion of the oligomer to the DHU form was >80% after 15 minutes.

### Photochemical Treatment of 74-Mer Oligomers 4 and 5

Photochemical conversion of bases such as 5-caC within longer oligomers (Oligomer 4 SEQ ID NO:4 and Oligomer 5 SEQ ID NO:5) under different reaction conditions was evaluated by the parallel preparation of multiple samples and pooled sequencing.

To each 500 μL Eppendorf were added the following components:

5 μL of 1 M, pH 4.5 aqueous sodium acetate buffer (final reaction concentration 100 mM),

10 μL photocatalyst, 0.5 mM stock solution in MBCN (final reaction concentrations 0.1 mM photocatalyst/20% MeCN),

1.76 μL neat 2-mercaptoethanol (final reaction concentration 0.5 M),

5-10 μL oligonucleotide aqueous stock solution (dependent on the concentration as supplied by the manufacturer; final reaction concentration 10 ng/μL),
Water (to 50 μL)

The solution was continually illuminated by blue LEDs (450 nm) in a PhotoRedOx Box (HepatoChem) under ambient atmosphere. Reactions were quenched after incubation for 0, 10 or 20 minutes, purified using Oligo Clean & Concentrate kits (Zymo) and diluted 100× with water. Library Preparation of 74-mer Oligomers 4 and 5

Samples were amplified and extended by PCR in reactions using KAPA HiFi Uracil+ polymerase and the primers PCR1_overhang_fwd (Primer 1) and PCR1_overhang_rev (Primer 2) using the following cycling conditions:

### PCR Programme

95° C. 3 mins
[98° C. 20 secs; 65° C. 15 secs; 72° C. 15 secs]×20
72° C. 1 min

PCR reactions were purified (Qiagen, MinElute Reaction Cleanup kit), quantified (Qubit, HS dsDNA kit) and diluted to 0.1 ng/μL.

Samples were secondly amplified and indexed by PCR in reactions using Q5 Ultra II polymerase (NEB) and the primers PCR2_universal_fwd (Primer 3) and one of PCR2_index_rev[i] (selected from Primer 4-01 to 4-27, SEQ ID NO:11 to SEQ ID NO:34) using the following cycling conditions:

### PCR Programme

98° C. 30 secs
[98° C. 10 secs; 65° C. 75 secs]×10
65° C. 1 min

PCR products were purified and quantified as above.

### Illumina Sequencing of 74-Mer Oligomers 4 and 5

Indexed samples were diluted to 4 nM, pooled and analysed by single-end sequencing using MiSeq Reagent v3 or nano v2 kits.

### Sequencing Results of 74-Mer Oligomers 4 and 5

Base transitions, including that of DHU generated from 5caC within synthetic oligonucleotides, were studied by sequencing of oligomers. The reaction conditions used and % conversion to T detected for oligomer samples and controls 1 to 8 are shown in Table 1.

Photoreactions all used 0.1 mM photocatalyst; samples 7 and 8 were untreated controls.

TABLE 1
Conditions used and comparison of conversion of 74-mer
Oligomers 4 and 5 vs untreated controls.

| | Oligomer | Oligo | Thiol | Reaction | T |
|---|---|---|---|---|---|
| Oligomer | (base at | conc./ | conc./ | time/ | conversion/ |
| Sample | position 41) | ng/μL | mM | min. | % |
| 1 | 4 (5caC) | 20 | 250 | 20 | 19.3 |
| 2 | 4 (5caC) | 20 | 250 | 40 | 20.3 |
| 3 | 4 (5caC) | 20 | 500 | 20 | 66.1 |
| 4 | 4 (5caC) | 10 | 250 | 20 | 31.5 |
| 5 | 4 (5caC) | 10 | 500 | 20 | 80.1 |
| 6 | 5 (C) | 20 | 250 | 20 | 2.6 |
| 7 | 4 (5caC) | 20 | — | — | 2.1 |
| 8 | 5 (C) | 20 | — | — | 2.2 |

In Oligomer Samples 1-5, varying amounts of conversion to T were detected at base 41 (corresponding to 5caC in the starting material). Unmodified Oligomer Sample 5 (with cytosine at position 41) exhibited a low rate of mutation to T, which was comparable to those of the two untreated controls, Oligomer Samples 7 and 8.

(This low baseline mutation rate observed for the synthetic oligo samples may be the product of various factors, such as errors during oligo synthesis, PCR amplification, and sequencing.)

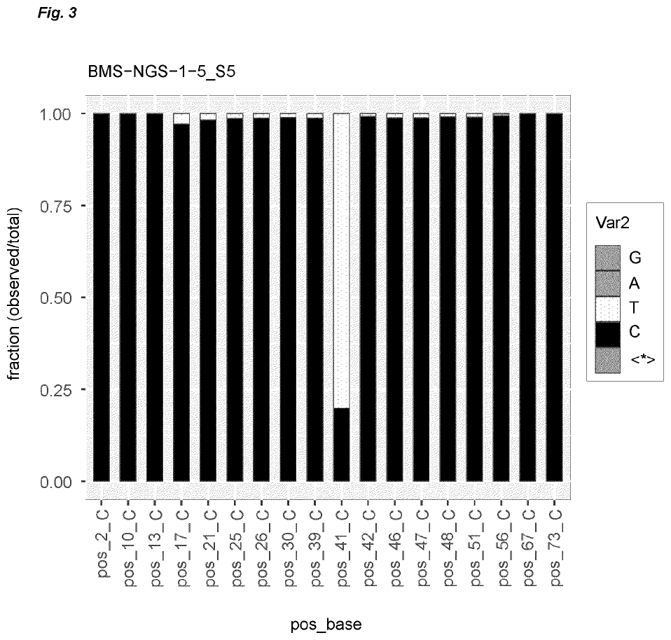

The conversion of cytosine residues detected within the 5caC Oligomer 4 was calculated and shown in FIG. 3. A strong C-to-T transition is detected by the method at position C_41 where 5caC is present in the starting material. In contrast, transitions at unmodified cytosine positions was minimal.

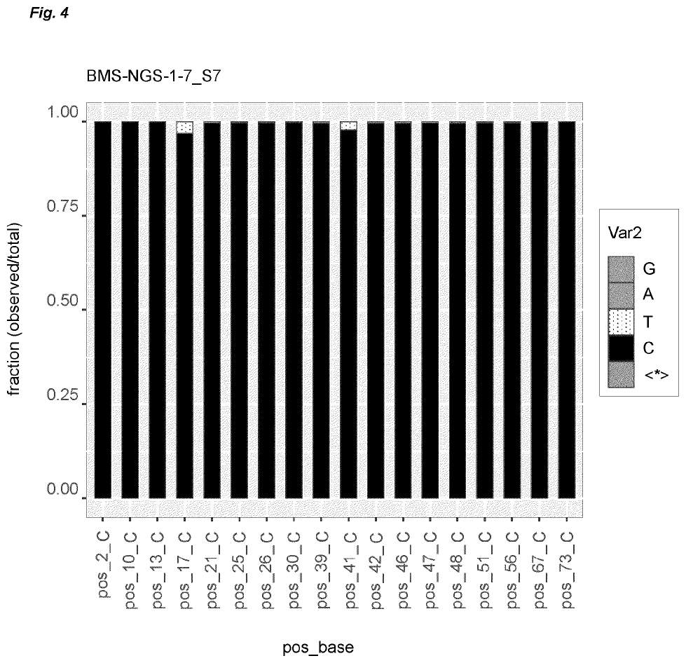

The conversion of unmodified bases was also calculated within each sample and shown in FIGS. 3 and 4. In addition to the control oligomers, this further demonstrates the selectivity of the treatment. Some low rate of background C-to-T conversion can be seen in FIG. 3. However, the analysis for sample 7 (untreated 5caC Oligomer 4) is provided for comparison at FIG. 4, which confirms that the conversion of unmodified bases, such as at the C_17 position, comes from sources other than the photochemical treatment.

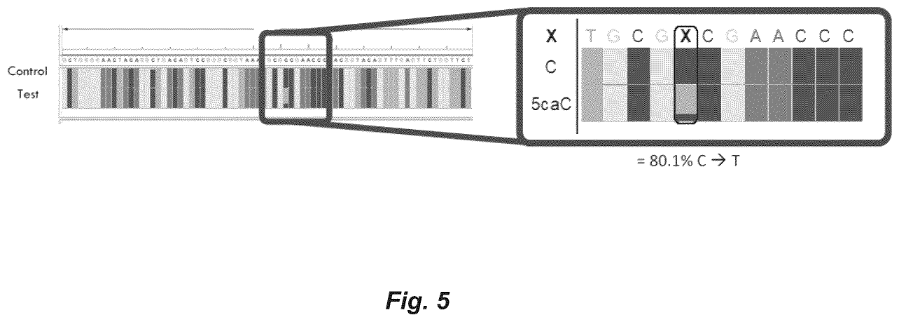

FIG. 5 shows a visualization of the proportion of bases detected in sequencing reads obtained from samples 5 and 6. When the 5caC Oligomer 4 sample was treated for 20 minutes with the photochemical conditions (bottom track), T was detected instead of C at the 5caC-modified position in 80.1% of reads. This is compared to 2.2% for the cytosine Oligomer 5 unmodified control (top track).

The effect of varying thiol, photocatalyst concentration and incubation time on the outcome in terms of C-to-T conversion was also explored. The results are shown in Table 2.

In all reactions 1 ng/μL oligonucleotide was used.

Through these optimization studies it can be see that the reaction is affected by the thiol concentration, and is also modestly affected by the incubation time.

TABLE 2
Comparison of conversion of Oligomer 4 under varying
treatment conditions.

| | Photocatalyst/ | Thiol | Reaction | T |
|---|---|---|---|---|
| Sample | mM | conc./mM | time/min. | conversion/% |
| 1_1 | 0.1 | 1000 | 10 | 59.0 |
| 1_2 | 0.1 | 1000 | 20 | 76.3 |
| 2_1 | 0.1 | 750 | 10 | 58.7 |
| 2_2 | 0.1 | 750 | 20 | 76.5 |
| 3_1 | 0.1 | 500 | 10 | 54.3 |
| 3_2 | 0.1 | 500 | 20 | 70.9 |
| 4_1 | 0.1 | 250 | 10 | 30.3 |
| 4_2 | 0.1 | 250 | 20 | 32.8 |
| 5_1 | 0.8 | 1000 | 10 | 64.3 |
| 5_2 | 0.8 | 1000 | 20 | 76.8 |
| 6_1 | 0.8 | 750 | 10 | 65.9 |
| 6_2 | 0.8 | 750 | 20 | 78.7 |
| 7_1 | 0.8 | 500 | 10 | 34.8 |
| 7_2 | 0.8 | 500 | 20 | 39.6 |
| 8_1 | 0.8 | 250 | 10 | 32.5 |
| 8_2 | 0.8 | 250 | 20 | 38.2 |
| 9_1 | 0.6 | 1000 | 10 | 64.1 |
| 9_2 | 0.6 | 1000 | 20 | 73.0 |
| 10_1 | 0.6 | 750 | 10 | 56.2 |
| 10_2 | 0.6 | 750 | 20 | 71.3 |
| 11_1 | 0.6 | 500 | 10 | 55.9 |
| 11_2 | 0.6 | 500 | 20 | 68.0 |
| 12_1 | 0.6 | 250 | 10 | 23.8 |
| 12_2 | 0.6 | 250 | 20 | 29.0 |

Taking the conditions used in sample 1_2 (Table 2) as representative reaction conditions, three repeat experiments were used to calculate the average rates of conversion to T in phototreated vs untreated controls. The % reads containing T at any position where 5caC, C, A or G was expected were calculated. The results are shown in Table 3.

TABLE 3
Average rates of conversion to T in
phototreated vs untreated controls.

| 5caC | Treated | Untreated |
|---|---|---|
| | 73.2 +/− 2.22% | 1.53 +/− 0.47% |
| C | 0.94 +/− 0% | 0.31 +/− 0% |
| A | 0.01 +/− 0% | 0.01 +/− 0% |
| G | 0.94 +/− 0.09% | 0.08 +/− 0% |

### Preparation of Methylated Lambda-DNA

Unmethylated Lambda-phage DNA (48.5 kpb) was acquired commercially. Artificial methylation was carried out twice using CpG methylase M.Sssl (Zymo), according to manufacturer's protocols. DNA was fragmented on a Covaris ultrasonicator to 200 base pairs. Complete methylation was validated by a single round of bisulfite sequencing (EZ DNA Methylation Lightning Kit, Zymo+MiSeq Reagent nano v2 Kit, Illumina).

Synthetic spike-ins were prepared with the following oligonucleotides acquired from ATDBio with PAGE purification:
100spike1_5caC: Oligomer 6 and
100spike2_5mC: Oligomer 7

These oligos were separately annealed with complementary DNA strands at 100 μM by heating to 95° C. for 3 minutes and cooling over 1 hour. Double-stranded spike-ins were mixed into methylated, fragmented Lambda-DNA (1:1:98 by mass).

### TET Oxidation of Methylated Lambda-DNA

Fragmented, CpG-methylated DNA was oxidised by TET2 taken from an EM-Seq Conversion Module (NEB), omitting the “oxidation enhancer” and “oxidation supplement” but otherwise following manufacturer's protocols. Reaction products were purified using 1.8× Ampure XP beads and eluted into nuclease-free water in preparation for the photochemical treatment. Recovery was between 70-90% of input material. Material was pooled and oxidised again, for a total of two treatments.

### Adaptor Ligation of Methylated Lambda-DNA

In order to carry out the photochemical reaction on single-stranded DNA, adaptor ligation (requiring double-stranded input) was carried out upstream of photochemical treatment

Samples were ligated with adaptors using xGen-UDI-UMI full-length adaptors (IDT) and KAPA HyperPrep kit (Roche) according to manufacturer's protocols, and purified using 0.8× Ampure XP beads.

### Photochemical Treatment of Oxidised Lambda-DNA

The 50 μL photoreactions were carried out on 100 ng quantities of prepared Lambda-DNA. In order to render the DNA single-stranded, reaction portions of library fragments from the previous step were diluted to the desired stock concentration, and heated to 95° C. for 5 minutes. The aliquots were briefly centrifuged and snap-frozen in a tube rack pre-cooled to −78° C. Pre-mixed reagents (buffered acetate solution, photocatalyst, thiol) were overlaid on the frozen samples, the contents of the tube mixed as the library stock melted, and incubated for up to 20 minutes using equipment described previously in synthetic oligonucleotide protocols. Reactions were purified using Oligo Clean & Concentrate columns.

### Control Treatment of Oxidised Lambda-DNA

Separately, control samples were bisulfite-treated to validate M.Sssl and TET2 efficiency (EZ DNA Methylation Lightning kit, Zymo).

### Library Preparation & Sequencing of Methylated Lambda-DNA

Samples were amplified using KAPA HiFi Uracil+ polymerase, pooled and sequenced according to standard procedures (paired-end/MiSeq nano v2 reagents).

### Sequencing Results of Methylated Lambda-DNA

The following five genomic sequencing libraries were prepared and the treatments used and results are summarized in Table 4.
Sample 1=sequencing control
Sample 2=bisulfite control (measures methylation of DNA)
Sample 3=TET-assisted bisulfite sequencing (TABS, measures TET oxidation of 5mC)
Samples 4 & 5=photochemistry tests, different reaction conditions.

TABLE 4
Genomic Samples 1 to 5.

| Genomic Sample | 1 | 2 | 3 | 4 | 5 |
|---|---|---|---|---|---|
| 2 × TET oxidation | — | — | + | + | + |
| | Library preparation | | | | |
| Bisulfite treatment | — | + | + | — | — |
| Photochemical | — | — | — | 10 mins | 20 mins |
| treatment | | | | | |
| mCpG detected[1] | [99.9%] | 84.4% | 7.4% | 82.5% | 74.3% |
| CpG-to-TpG | [0.1%] | — | — | 17.5% | 25.7% |
| conversions[2] | | | | | |
| Photochemical 5caC- | | | | 22.7% | 33.4% |
| to-T conversion rate | | | | | |

Photoreactions both used 1 M thiol and 0.1 mM photocatalyst.

In the table, “+” indicates that the specified treatment was carried out the sample, and “−” indicates the absence of the specified treatment or that the results were not applicable or not analysed.
^{[1]} Processed as 3-letter alphabet using Bismark.
^{[2]} Processed as 4-letter alphabet using custom analysis pipeline.

Up until the library preparation step described above, material was pooled between each step such that the fraction of DNA methylated/TET-oxidised would be identical between samples after indexing & before chemical treatments. Libraries were prepared including UMIs to remove PCR duplicates.

The results are shown in Tables 4 and 5.

Bisulfite sequencing (Genomic Sample 2) indicated that 84.4% of CpG sites within the genome had been artificially methylated, i.e. 15.6% of CpG sites remained unmodified (Table 4).

TET-assisted bisulfite sequencing (Genomic Sample 3) indicated that after TET oxidation, methylation levels fell from 84.4% to 7.4%-77% of total CpG sites were converted from mCpG to oxCpG by TET (Table 4). This can also be expressed as: 91.2% of 5mC modifications were oxidised to 5fC/5caC.

A 10-minute photoincubation reaction (Genomic Sample 4) resulted in 17.5% C-to-T conversion at CpG sites. Since the chemistry leaves unmodified cytosines unconverted, C-to-T transitions can only result from successive oxidation & photochemical deamination at methylated sites. Considering the photochemical conversion alone (as a fraction of oxCpGs measured to be present), 17.5% over 77% (from TABS)=22.7% conversion rate of 5caC to DHU.

A 20-minute photoincubation reaction (Genomic Sample 5) resulted in 25.7% conversion at CpG sites. Interpreting as with sample 4, 33.4% of oxCpG sites were converted to DHU in the photoreaction.

The conversion efficiency and sequencing coverage across different CpG contexts is shown in Table 5.

TABLE 5
Summary of conversion efficiency and sequencing coverage across the four genomic
CGN contexts.

| | | Number | Number of | Number of | Number of | |
|---|---|---|---|---|---|---|
| | | of | genomic | methylated | unmodified | Detected |
| | Specific | genomic | positions | bases (total C | bases (total C | modification |
| Library | context | positions | covered | read as C) | read as T) | rate (%) |
| 1 | CGN | 6249 | 6249 | 524674 | 332 | 99.94 |
| | CGA | 1217 | 1217 | 100634 | 64 | 99.94 |
| | CGC | 1737 | 1737 | 144523 | 96 | 99.93 |
| | CGG | 1850 | 1850 | 152630 | 98 | 99.94 |
| | CGT | 1445 | 1445 | 126887 | 74 | 99.94 |
| 2 | CGN | 6249 | 6148 | 245168 | 45252 | 84.42 |
| | CGA | 1217 | 1203 | 64494 | 11798 | 84.54 |
| | CGC | 1737 | 1715 | 57749 | 11155 | 83.81 |
| | CGG | 1850 | 1813 | 66070 | 11882 | 84.76 |
| | CGT | 1445 | 1417 | 56855 | 10417 | 84.52 |
| 3 | CGN | 6249 | 3936 | 10728 | 134931 | 7.37 |
| | CGA | 1217 | 84 | 1315 | 40392 | 3.15 |
| | CGC | 1737 | 1119 | 4309 | 30725 | 12.30 |
| | CGG | 1850 | 1048 | 3743 | 30149 | 11.04 |
| | CGT | 1445 | 928 | 1361 | 33665 | 3.89 |
| | | | | Number of | Number of | Detected |
| | | | | methylated | unconverted | modification |
| | | | | bases detected | bases (total C | rate (%) |
| | | | | (total C read | read as C) | |
| | | | | as T) | | |
| 4 | CGN | 6249 | 6249 | 87796 | 414979 | 17.46 |
| | CGA | 1217 | 1217 | 21411 | 78732 | 21.38 |
| | CGC | 1737 | 1737 | 21712 | 113488 | 16.06 |
| | CGG | 1850 | 1850 | 18517 | 129165 | 12.54 |
| | CGT | 1445 | 1445 | 26156 | 93594 | 21.84 |
| | | Number | Number of | Number of | Number of | |
| | | of | genomic | methylated | unmodified | Detected |
| | Specific | genomic | positions | bases (total C | bases (total C | modification |
| Library | context | positions | covered | read as C) | read as T) | rate (%) |
| 5 | CGN | 6249 | 6249 | 141650 | 409807 | 25.69 |
| | CGA | 1217 | 1217 | 35148 | 76611 | 31.45 |
| | CGC | 1737 | 1737 | 34850 | 115282 | 23.21 |
| | CGG | 1850 | 1850 | 31255 | 128423 | 19.57 |
| | CGT | 1445 | 1445 | 40397 | 89491 | 31.10 |

### Methylated Lambda-DNA—Ophmising Conditions

In this set of experiments, methylated DNA sequencing libraries were treated as follows:
Genomic DNA was sonicated to fragments 250 base pairs long
TET oxidation was carried out three times in total
As described in the genomic experiments above, after methylation, TET oxidation and library preparation were carried out, aliquots were heated to 95° C. and snap-frozen at −78° C., before immediately being used in photoreactions.

Sequencing data from bisulphite-treated libraries were analysed by alignment and processing using Bismark and/or the Astair tool (C-to-T mode) developed for use with TAPS as described in Uu et al.

Sequencing data from photochemically-treated libraries were analysed using the canonical, four-letter DNA alphabet with the Astair (mC-to-T mode).

The following libraries were prepared for sequencing. These are summarised in Table 6.
Sample 1=bisulphite control (methylation of DNA)
Sample 2=TABS (TET oxidation of 5mC)
Samples 3 to 9=photochemistry tests with different reaction conditions.

TABLE 6
Methylated Lambda-DNA samples.

| Library | 1 | 2 | 3 | 4 | 5 | 6 | 7 | 8 | 9 |
|---|---|---|---|---|---|---|---|---|---|
| 3x TET oxidation | − | + | + | + | + | + | + | + | + |
| | Library preparation | | | | | | | | |
| Chemical | BS | BS | PC | PC | PC | PC | PC | PC | PC |
| treatment | | | | | | | | | |
| Treatment length | − | − | 10 | 20 | 10 | 20 | 10 | 10 | 10 |
| (mins) | | | | | | | | | |
| Thiol conc. (M) | − | − | 1 | 1 | 2.5 | 2.5 | 1 | 1 | 1 |
| PC conc (mM) | − | − | 0.1 | 0.1 | 0.1 | 0.1 | 0.25 | 0.1 | 0.1 |
| DNA input (ng) | 100 | 100 | 200 | 200 | 200 | 200 | 200 | 100 | 50 |
| mCpGs protected | 95.0% | 11.8% | | | | | | | |
| during BS-Seq^{[1]} | | | 54.9% | 60.2% | 23.1% | 39.2% | 57.3% | 53.5% | 55.9% |
| CpG-to-TpG | | | | | | | | | |
| conversion rate^{[2]} | | | 66.1% | 72.4% | 27.8% | 47.2% | 68.9% | 64.4% | 67.3% |
| oxC-to-T | − | − | | | | | | | |
| conversion rate^{[3]} | | | | | | | | | |

In Table 6, “+” indicates that the specified treatment was carried out the sample, and “−” indicates the absence of the specified treatment or that the results were not applicable or not analysed.
^{[1]} Processed as 3-letter alphabet using C-to-T mode.
^{[2]} Processed as 4-letter alphabet using mC-to-T mode.
^{[3]} Estimated as a percentage of oxCs converted to T.
oxC is defined as 5fC and 5caC [& not 5hmC].

Material was pooled between each step up until the library preparation step. Therefore, each library is believed to contain the same proportion of C, 5mC and oxidised Cs going into chemical treatments.

Bisulphite sequencing (Library 1) determined that 95.0% of CpG sites within the genome had been methylated. TET-assisted bisulfite sequencing (Library 2) indicated that after TET oxidation, 11.8% of CpG sites contained 5mC or 5hmC. The efficiency of TET oxidation of 5mC to oxC was 87.5%.

The rate of base conversion in photochemical reactions was determined relative to the estimated amount of oxC present:

$$\% conversion = \frac{Observed C to {T}_{Library N}}{( \% {Methylation}_{Library 1} ) - ( \% {Methylation}_{Library 2} )}$$

For example, in Library 3, 54.9% of all CpGs were converted to TpGs. Since 83.2% of all CpGs were present in the 5fC/5caC state, the photochemical conversion itself was at least 66.1% efficient. This is believed to be a minimum estimate which assumes that 5caC is the only product of TET oxidation and that 5fC is not converted by the photoreaction. The proportion of 5fC:5caC produced by TET was not determined experimentally here, so it is possible that the true rate of 5caC conversion may be higher.

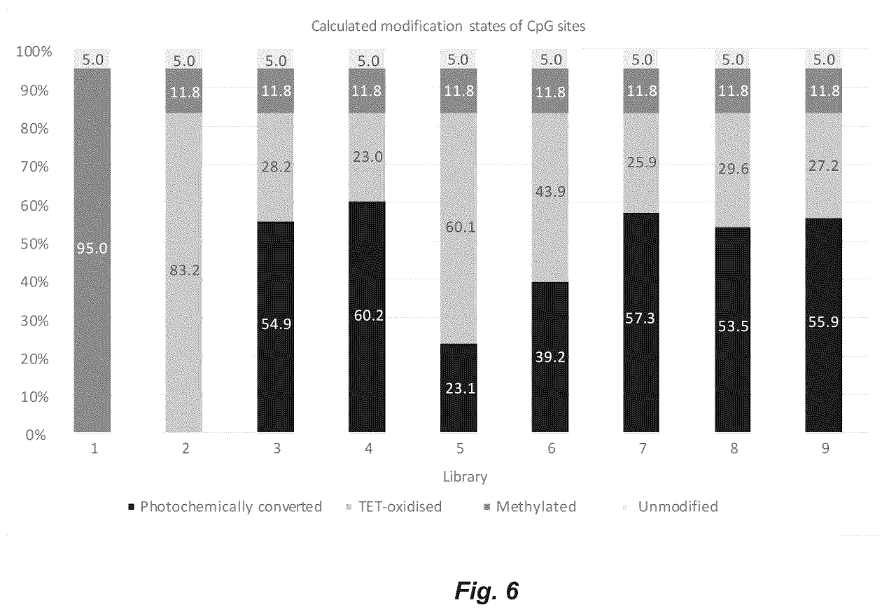

By comparing the libraries, the proportion of CpG sites in each modification state can be estimated. This is shown in FIG. 6.

For each library shown in FIG. 6:

$$\% photochemical conversion = \frac{photochemically converted}{photochemically converted + TET -oxidised}$$

Data analysis for Libraries 3 to 9 was carried out using a four-letter alphabet. This is an advantage of the present method as unmodified C being is unreactive towards the photochemistry and therefore sequence complexity is retained.

An analysis of step efficiency in different CGN contexts is shown in Table 7.

TABLE 7
Summary of conversion efficiency across the four
genomic CGN contexts fo reach enzymatic step and
under different photochemical reaction conditions.

| | | Detected | Calculated |
|---|---|---|---|
| | Specific | overall | step efficiency |
| Library | context | efficiency (%) | (%) |
| | mC introduced by M.Sssl | | |
| 1 | CGN | 95.00 | 95.00 |
| | CGA | 95.04 | 95.04 |
| | CGC | 94.58 | 94.58 |
| | CGG | 95.26 | 95.26 |
| | CGT | 95.11 | 95.11 |
| | mC remaining after TET oxidation | | |
| 2 | CGN | 11.85 | 87.53 |
| | CGA | 4.47 | 95.30 |
| | CGC | 20.39 | 78.44 |
| | CGG | 18.31 | 80.78 |
| | CGT | 6.70 | 92.95 |
| | Photochemical mC-to-Ts | | |
| 3 | CGN | 54.95 | 66.08 |
| | CGA | 64.22 | 70.90 |
| | CGC | 47.70 | 64.29 |
| | CGG | 48.26 | 62.72 |
| | CGT | 62.41 | 70.59 |
| 4 | CGN | 60.19 | 72.38 |
| | CGA | 70.10 | 77.40 |
| | CGC | 51.78 | 69.79 |
| | CGG | 54.10 | 70.31 |
| | CGT | 66.87 | 75.64 |
| 5 | CGN | 23.09 | 27.77 |
| | CGA | 28.02 | 30.94 |
| | CGC | 20.87 | 28.12 |
| | CGG | 17.69 | 22.98 |
| | CGT | 27.43 | 31.03 |
| 6 | CGN | 39.22 | 47.16 |
| | CGA | 47.80 | 52.78 |
| | CGC | 33.76 | 45.50 |
| | CGG | 31.58 | 41.03 |
| | CGT | 45.42 | 51.38 |
| 7 | CGN | 57.26 | 68.86 |
| | CGA | 67.33 | 74.34 |
| | CGC | 49.40 | 66.58 |
| | CGG | 50.11 | 65.11 |
| | CGT | 65.14 | 73.69 |
| 8 | CGN | 53.52 | 64.36 |
| | CGA | 62.91 | 69.46 |
| | CGC | 46.04 | 62.05 |
| | CGG | 45.60 | 59.25 |
| | CGT | 62.08 | 70.21 |
| 9 | CGN | 55.93 | 67.26 |
| | CGA | 65.45 | 72.27 |
| | CGC | 47.81 | 64.44 |
| | CGG | 48.54 | 63.08 |
| | CGT | 64.37 | 72.81 |

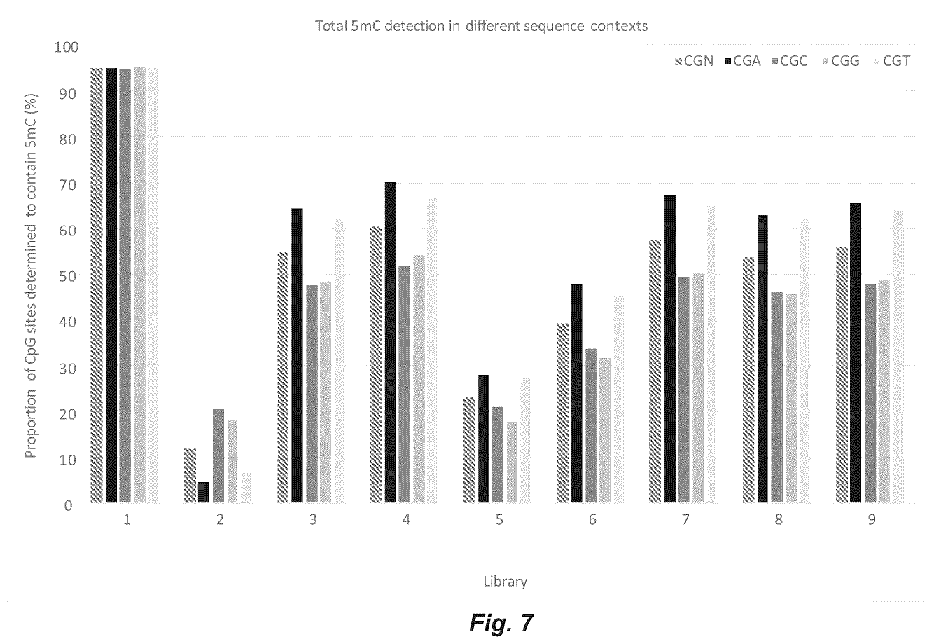

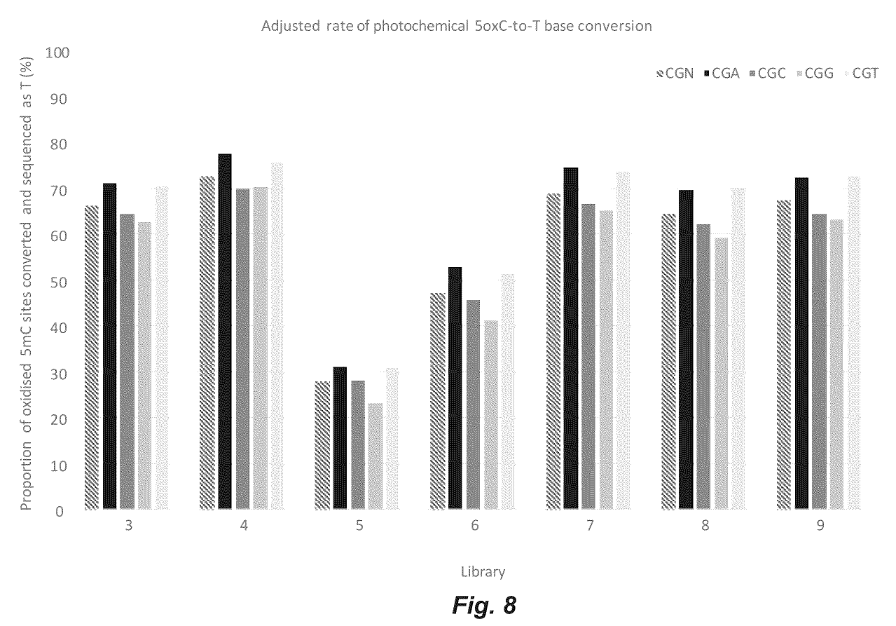

The proportion of CpG sites determined to contain 5mC in each of the libraries is shown in FIG. 7 (total sites detected) and FIG. 8 (normalised against observed TET oxidation efficiency).

The sequencing results show that M.Sssl methylated the four CGN sequence contexts evenly. 5mC is detected using the present method across the different CGN sequence contexts with good overall detection efficiency and step efficiency.

### Model Sequences

Oligomer 1-10 nucleotide long single stranded DNA model [5caC position
indicated by bold, italics and underlined]
(SEQ ID NO: 1)
CTTAC [symbol] CTGA
Oligomer 2-10 nucleotide long single stranded DNA model [5caC position
indicated by bold, italics and underlined]
(SEQ ID NO: 2)
TCAG [symbol] GTAAG
Oligomer 3-10 nucleotide long single stranded DNA model
(SEQ ID NO: 3)
TCAGGGTAAG
Oligomer 4-74 nucleotide long DNA model [5caC position indicated by bold,
italics and underlined]
(SEQ ID NO: 4)
GCTGGGGAACTACAGGCTGACAGTCCGGGGGGTAAATGCG [symbol] CGAACCCGACGGTACAGTTTG
AGTTCTGGTTCT
Oligomer 5-74 nucleotide long DNA model
(SEQ ID NO: 5)
GCTGGGGAACTACAGGCTGACAGTCCGGGCGGTAAATGCGCCGAACCCGACGGTACAGTTTG
AGTTCTGGTTCT
Oligomer 6-74 nucleotide long DNA model [5caC position indicated by
bold, italics and underlined]
(SEQ ID NO: 6)
GCTGGGGAACTACAGGCTGACAGTACGTGCCGTAAATGCG [symbol] GTAGTCCGTCAGTACCGATGC
TGAACAAGTCGATGCAGTACAGTTTGAGTTCTGGTTCT
Oligomer 7-74 nucleotide long DNA model [5mC position indicated by
bold, italics and underlined]
(SEQ ID NO: 7)
GCTGGGGAACTACAGGCTCACTTGCGTGTAGATTATGTAGGGCG [symbol] GAAATGCAGGAGAAGTT
CTCGACCTTCTCGTGGGTACAGTTTGAGTTCTGGTTCT
Primer Sequences
Primer 1-PCR1_overhang_fwd
(SEQ ID NO: 8)
ACACTCTTTCCCTACACGACGCTCTTCCGATCTGCTGGGGAACTACAGG
Primer 2-PCR1_overhang_rev
(SEQ ID NO: 9)
GTGACTGGAGTTCAGACGTGTGCTCTTCCGATCTAGAACCAGAACTCAAACTGTA
Primer 3-PCR2_universal_fwd
(SEQ ID NO: 10)
AATGATACGGCGACCACCGAGATCTACACTCTTTCCCTACACGACGCTCTTCCGATCT
Primer 4-01-PCR2_index_rev1
(SEQ ID NO: 11)
CAAGCAGAAGACGGCATACGAGATCGTGATGTGACTGGAGTTCAGACGTGTGCTCTTCCGATC
T
Primer 4-02-PCR2_index_rev2
(SEQ ID NO: 12)
CAAGCAGAAGACGGCATACGAGATACATCGGTGACTGGAGTTCAGACGTGTGCTCTTCCGATC
T
Primer 4-03-PCR2_index_rev3
(SEQ ID NO: 13)
CAAGCAGAAGACGGCATACGAGATGCCTAAGTGACTGGAGTTCAGACGTGTGCTCTTCCGATC
T
Primer 4-04-PCR2_index_rev4
(SEQ ID NO: 14)
CAAGCAGAAGACGGCATACGAGATTGGTCAGTGACTGGAGTTCAGACGTGTGCTCTTCCGATC
T
Primer 4-05-PCR2_index_rev5
(SEQ ID NO: 15)
CAAGCAGAAGACGGCATACGAGATCACTGTGTGACTGGAGTTCAGACGTGTGCTCTTCCGATC
T
Primer 4-06-PCR2_index_rev6
(SEQ ID NO: 16)
CAAGCAGAAGACGGCATACGAGATATTGGCGTGACTGGAGTTCAGACGTGTGCTCTTCCGATC
T
Primer 4-07-PCR2_index_rev7
(SEQ ID NO: 17)
CAAGCAGAAGACGGCATACGAGATGATCTGGTGACTGGAGTTCAGACGTGTGCTCTTCCGATC
T
Primer 4-08-PCR2_index_rev8
(SEQ ID NO: 18)
CAAGCAGAAGACGGCATACGAGATTCAAGTGTGACTGGAGTTCAGACGTGTGCTCTTCCGATC
T
Primer 4-09-PCR2_index_rev9
(SEQ ID NO: 19)
CAAGCAGAAGACGGCATACGAGATCTGATCGTGACTGGAGTTCAGACGTGTGCTCTTCCGATC
T
Primer 4-10-PCR2_index_rev10
(SEQ ID NO: 20)
CAAGCAGAAGACGGCATACGAGATAAGCTAGTGACTGGAGTTCAGACGTGTGCTCTTC
CGATCT
Primer 4-11-PCR2_index_rev11
(SEQ ID NO: 21)
CAAGCAGAAGACGGCATACGAGATGTAGCCGTGACTGGAGTTCAGACGTGTGCTCTTCCGAT
CT
Primer 4-12-PCR2_index_rev12
(SEQ ID NO: 22)
CAAGCAGAAGACGGCATACGAGATTACAAGGTGACTGGAGTTCAGACGTGTGCTCTTCCGATC
T
Primer 4-13-PCR2_index_rev13
(SEQ ID NO: 23)
CAAGCAGAAGACGGCATACGAGATTTGACTGTGACTGGAGTTCAGACGTGTGCTCTTCCGATC
T
Primer 4-14-PCR2_index_rev14
(SEQ ID NO: 24)
CAAGCAGAAGACGGCATACGAGATGGAACTGTGACTGGAGTTCAGACGTGTGCTCTTCCGATC
T
Primer 4-15-PCR2_index_rev15
(SEQ ID NO: 25)
CAAGCAGAAGACGGCATACGAGATTGACATGTGACTGGAGTTCAGACGTGTGCTCTTCCGATC
T
Primer 4-16-PCR2_index_rev16
(SEQ ID NO: 26)
CAAGCAGAAGACGGCATACGAGATGGACGGGTGACTGGAGTTCAGACGTGTGCTCTTCCGAT
CT
Primer 4-18-PCR2_index_rev18
(SEQ ID NO: 27)
CAAGCAGAAGACGGCATACGAGATGCGGACGTGACTGGAGTTCAGACGTGTGCTCTTCCGAT
CT
Primer 4-19-PCR2_index_rev19
(SEQ ID NO: 28)
CAAGCAGAAGACGGCATACGAGATTTTCACGTGACTGGAGTTCAGACGTGTGCTCTTCCGATC
T
Primer 4-20-PCR2_index_rev20
(SEQ ID NO: 29)
CAAGCAGAAGACGGCATACGAGATGGCCACGTGACTGGAGTTCAGACGTGTGCTCTTCCGAT
CT
Primer 4-21-PCR2_index_rev21
(SEQ ID NO: 30)
CAAGCAGAAGACGGCATACGAGATCGAAACGTGACTGGAGTTCAGACGTGTGCTCTTCCGATC
T
Primer 4-22-PCR2_index_rev22
(SEQ ID NO: 31)
CAAGCAGAAGACGGCATACGAGATCGTACGGTGACTGGAGTTCAGACGTGTGCTCTTCCGAT
CT
Primer 4-23-PCR2_index_rev23
(SEQ ID NO: 32)
CAAGCAGAAGACGGCATACGAGATCCACTCGTGACTGGAGTTCAGACGTGTGCTCTTCCGATC
T
Primer 4-25-PCR2_index_rev25
(SEQ ID NO: 33)
CAAGCAGAAGACGGCATACGAGATATCAGTGTGACTGGAGTTCAGACGTGTGCTCTTCCGATC
T
Primer 4-27-PCR2_index_rev27
(SEQ ID NO: 34)
CAAGCAGAAGACGGCATACGAGATAGGAATGTGACTGGAGTTCAGACGTGTGCTCTTCCGATC
T

### REFERENCES

All documents mentioned in this specification are incorporated herein by reference in their entirety.
Bentley et al. Nature 2008, 456, 53
Danielson et al. Polymers 2018, 10, 741
Deaton et al. Genes Dev. 2011, 25, 1010
Liu et al. Nature Biotechnology 2019, 37, 424
Sanger et al. PNAS 1977, 74, 5463
WO 2019/136413
WO 2013/017853

1. A method of identifying a modified cytosine residue in a sample nucleotide sequence, the method comprising;
(i) providing a population of polynucleotides which comprise the sample nucleotide sequence,
(ii) reducing and deaminating a 5-methylcytosine (5mC) residue within the population,
(iii) sequencing the polynucleotides in the population or derivatives thereof following step (ii) to produce a treated nucleotide sequence, and;
(iv) identifying the residue in the treated nucleotide sequence which corresponds to a modified cytosine residue in the sample nucleotide sequence.

2. The method of claim 1, wherein the reduction is the reduction of the C5-C6 bond in a modified cytosine residue, and the deamination is the loss of the amino group at the C4 position in a modified cytosine residue, which is replaced with hydroxyl.

3. The method of claim 1, wherein the presence of a thymine residue in the treated nucleotide sequence is indicative that the modified cytosine residue in the sample nucleotide sequence is 5-methylcytosine (5mC).

4. A method of identifying 5-methylcytosine (5mC) or 5-carboxylcytosine (5caC) in a sample nucleotide sequence, the method comprising:
(i) providing a population of polynucleotides which comprise the sample nucleotide sequence,
(ii) treating the population with a radical initiator together with a nucleophile,
(iii) sequencing the polynucleotides in the population or derivatives thereof following step (ii) to produce a treated nucleotide sequence, and;
(iv) identifying the residue in the treated nucleotide sequence which corresponds to a modified cytosine residue in the sample nucleotide sequence,
wherein the presence of a thymine residue in the treated nucleotide sequence is indicative that the modified cytosine residue in the sample nucleotide sequence is 5-methylcytosine (5mC) or 5-carboxylcytosine (5caC).

5. A method of identifying a modified cytosine residue in a sample nucleotide sequence, the method comprising:
(i) providing a population of polynucleotides which comprise the sample nucleotide sequence,
(ii) oxidising a first portion of said population,
(iii) treating the oxidised first portion of said population with a radical initiator together with a nucleophile, sequencing the polynucleotides in the first portions of the population or derivatives thereof following steps ii) and iii) to produce a first nucleotide sequence, and;
(iv) identifying the residue in the first nucleotide sequences which corresponds to a modified cytosine residue in the sample nucleotide sequence.

6. The method of claim 5, wherein in step (ii), the oxidation is the oxidation of a C5 methyl group in a modified cytosine residue.

7. The method of claim 5, wherein the oxidation is an oxidation by a ten-eleven-translocation (TET) oxygens.

8. A method for modifying a polynucleotide, the method comprising converting a 5-methylcytosine (5mC) residue in a polynucleotide to a dihydrothymine (DHT) residue by contacting the 5mc residue with a radical initiator and a nucleophile compound.

9. (canceled)

10. The method according claim 8, wherein the radical initiator is a transition metal photocatalyst.

11. The method according to claim 10, wherein the transition metal photocatalyst.

12. The method according to claim 11, wherein the transition metal photocatalyst comprises [Ir(dF(CF₃)ppy)₂(dtbpy)]+.

13. The method according to claim 1, wherein the nucleophile is a thiol compound, and/or the disulfide form thereof.

14. The method according to claim 13, wherein the thiol compound has one, two or three thiol groups, and the thiol compound has one, two or three hydroxyl groups.

15. The method according to claim 14, wherein the thiol is an alkyl thiol.

16. The method according to claim 13, wherein the thiol is mercaptoethanol.

17. The method according to claim 5, wherein, in step (ii), the first portion of the polynucleotide is treated with a ten-eleven translocation (TET) dioxygenase.

18. The method according to claim 1, wherein the polynucleotides are genomic DNA.

19. The method according to claim 1, wherein the polynucleotides are RNA.

20. The method according to claim 19, wherein the RNA is genomic RNA, mRNA, tRNA, rRNA or non-coding RNA.

21. (canceled)

22. (canceled)

23. (canceled)

24. A kit for identifying a modified cytosine residue comprising:
(a) a radical initiator, comprising [Ir(dF(CF₃)ppy)₂(dtbpy)]Cl; and
(b) a nucleophile, comprising a thiol compound.

25-50. (canceled)


SEQUENCE LISTING (36 sequences)

SEQ ID NO: 1 - DNA, 10 bp, synthetic construct
  misc_feature 1..10: note = synthetic construct
  source 1..10: mol_type = other DNA
  modified_base 6: mod_base = OTHER; note = 5-carboxylcytosine
cttaccctga

SEQ ID NO: 2 - DNA, 10 bp, synthetic construct
  misc_feature 1..10: note = synthetic construct
  source 1..10: mol_type = other DNA
  modified_base 5: mod_base = OTHER; note = 5-carboxylcytosine
tcagcgtaag

SEQ ID NO: 3 - DNA, 10 bp, synthetic construct
  misc_feature 1..10: note = synthetic construct
  source 1..10: mol_type = other DNA
tcagggtaag

SEQ ID NO: 4 - DNA, 74 bp, synthetic construct
  misc_feature 1..74: note = synthetic construct
  source 1..74: mol_type = other DNA
  modified_base 41: mod_base = OTHER; note = 5-carboxylcytosine
gctggggaactacaggctgacagtccgggcggtaaatgcgccgaacccgacggtacagtttgagttctggttct

SEQ ID NO: 5 - DNA, 74 bp, synthetic construct; identical to SEQ ID NO: 4
  misc_feature 1..74: note = synthetic construct
  source 1..74: mol_type = other DNA

SEQ ID NO: 6 - DNA, 100 bp, synthetic construct
  misc_feature 1..100: note = synthetic construct
  source 1..100: mol_type = other DNA
  modified_base 41: mod_base = OTHER; note = 5-carboxylcytosine
gctggggaactacaggctgacagtacgtgccgtaaatgcgcgtagtccgtcagtaccgatgctgaacaagtcgatgcagtacagtttgagttctggttct

SEQ ID NO: 7 - DNA, 100 bp, synthetic construct
  misc_feature 1..100: note = synthetic construct
  source 1..100: mol_type = other DNA
  modified_base 45: mod_base = OTHER; note = 5-methylcytosine
gctggggaactacaggctcacttgcgtgtagattatgtagggcgcgaaatgcaggagaagttctcgaccttctcgtgggtacagtttgagttctggttct

SEQ ID NO: 8 - DNA, 49 bp, synthetic construct
  misc_feature 1..49: note = synthetic construct
  source 1..49: mol_type = other DNA
acactctttccctacacgacgctcttccgatctgctggggaactacagg

SEQ ID NO: 9 - DNA, 55 bp, synthetic construct
  misc_feature 1..55: note = synthetic construct
  source 1..55: mol_type = other DNA
gtgactggagttcagacgtgtgctcttccgatctagaaccagaactcaaactgta

SEQ ID NO: 10 - DNA, 58 bp, synthetic construct
  misc_feature 1..58: note = synthetic construct
  source 1..58: mol_type = other DNA
aatgatacggcgaccaccgagatctacactctttccctacacgacgctcttccgatct

SEQ ID NO: 11 - DNA, 64 bp, synthetic construct
  misc_feature 1..64: note = synthetic construct
  source 1..64: mol_type = other DNA
caagcagaagacggcatacgagatcgtgatgtgactggagttcagacgtgtgctcttccgatct

SEQ ID NO: 12 - DNA, 64 bp, synthetic construct
  misc_feature 1..64: note = synthetic construct
  source 1..64: mol_type = other DNA
caagcagaagacggcatacgagatacatcggtgactggagttcagacgtgtgctcttccgatct

SEQ ID NO: 13 - DNA, 64 bp, synthetic construct
  misc_feature 1..64: note = synthetic construct
  source 1..64: mol_type = other DNA
caagcagaagacggcatacgagatgcctaagtgactggagttcagacgtgtgctcttccgatct

SEQ ID NO: 14 - DNA, 64 bp, synthetic construct
  misc_feature 1..64: note = synthetic construct
  source 1..64: mol_type = other DNA
caagcagaagacggcatacgagattggtcagtgactggagttcagacgtgtgctcttccgatct

SEQ ID NO: 15 - DNA, 64 bp, synthetic construct
  misc_feature 1..64: note = synthetic construct
  source 1..64: mol_type = other DNA
caagcagaagacggcatacgagatcactgtgtgactggagttcagacgtgtgctcttccgatct

SEQ ID NO: 16 - DNA, 64 bp, synthetic construct
  misc_feature 1..64: note = synthetic construct
  source 1..64: mol_type = other DNA
caagcagaagacggcatacgagatattggcgtgactggagttcagacgtgtgctcttccgatct

SEQ ID NO: 17 - DNA, 64 bp, synthetic construct
  misc_feature 1..64: note = synthetic construct
  source 1..64: mol_type = other DNA
caagcagaagacggcatacgagatgatctggtgactggagttcagacgtgtgctcttccgatct

SEQ ID NO: 18 - DNA, 64 bp, synthetic construct
  misc_feature 1..64: note = synthetic construct
  source 1..64: mol_type = other DNA
caagcagaagacggcatacgagattcaagtgtgactggagttcagacgtgtgctcttccgatct

SEQ ID NO: 19 - DNA, 64 bp, synthetic construct
  misc_feature 1..64: note = synthetic construct
  source 1..64: mol_type = other DNA
caagcagaagacggcatacgagatctgatcgtgactggagttcagacgtgtgctcttccgatct

SEQ ID NO: 20 - DNA, 64 bp, synthetic construct
  misc_feature 1..64: note = synthetic construct
  source 1..64: mol_type = other DNA
caagcagaagacggcatacgagataagctagtgactggagttcagacgtgtgctcttccgatct

SEQ ID NO: 21 - DNA, 64 bp, synthetic construct
  misc_feature 1..64: note = synthetic construct
  source 1..64: mol_type = other DNA
caagcagaagacggcatacgagatgtagccgtgactggagttcagacgtgtgctcttccgatct

SEQ ID NO: 22 - DNA, 64 bp, synthetic construct
  misc_feature 1..64: note = synthetic construct
  source 1..64: mol_type = other DNA
caagcagaagacggcatacgagattacaaggtgactggagttcagacgtgtgctcttccgatct

SEQ ID NO: 23 - DNA, 64 bp, synthetic construct
  misc_feature 1..64: note = synthetic construct
  source 1..64: mol_type = other DNA
caagcagaagacggcatacgagatttgactgtgactggagttcagacgtgtgctcttccgatct

SEQ ID NO: 24 - DNA, 64 bp, synthetic construct
  misc_feature 1..64: note = synthetic construct
  source 1..64: mol_type = other DNA
caagcagaagacggcatacgagatggaactgtgactggagttcagacgtgtgctcttccgatct

SEQ ID NO: 25 - DNA, 64 bp, synthetic construct
  misc_feature 1..64: note = synthetic construct
  source 1..64: mol_type = other DNA
caagcagaagacggcatacgagattgacatgtgactggagttcagacgtgtgctcttccgatct

SEQ ID NO: 26 - DNA, 64 bp, synthetic construct
  misc_feature 1..64: note = synthetic construct
  source 1..64: mol_type = other DNA
caagcagaagacggcatacgagatggacgggtgactggagttcagacgtgtgctcttccgatct

SEQ ID NO: 27 - DNA, 64 bp, synthetic construct
  misc_feature 1..64: note = synthetic construct
  source 1..64: mol_type = other DNA
caagcagaagacggcatacgagatgcggacgtgactggagttcagacgtgtgctcttccgatct

SEQ ID NO: 28 - DNA, 64 bp, synthetic construct
  misc_feature 1..64: note = synthetic construct
  source 1..64: mol_type = other DNA
caagcagaagacggcatacgagattttcacgtgactggagttcagacgtgtgctcttccgatct

SEQ ID NO: 29 - DNA, 64 bp, synthetic construct
  misc_feature 1..64: note = synthetic construct
  source 1..64: mol_type = other DNA
caagcagaagacggcatacgagatggccacgtgactggagttcagacgtgtgctcttccgatct

SEQ ID NO: 30 - DNA, 64 bp, synthetic construct
  misc_feature 1..64: note = synthetic construct
  source 1..64: mol_type = other DNA
caagcagaagacggcatacgagatcgaaacgtgactggagttcagacgtgtgctcttccgatct

SEQ ID NO: 31 - DNA, 64 bp, synthetic construct
  misc_feature 1..64: note = synthetic construct
  source 1..64: mol_type = other DNA
caagcagaagacggcatacgagatcgtacggtgactggagttcagacgtgtgctcttccgatct

SEQ ID NO: 32 - DNA, 64 bp, synthetic construct
  misc_feature 1..64: note = synthetic construct
  source 1..64: mol_type = other DNA
caagcagaagacggcatacgagatccactcgtgactggagttcagacgtgtgctcttccgatct

SEQ ID NO: 33 - DNA, 64 bp, synthetic construct
  misc_feature 1..64: note = synthetic construct
  source 1..64: mol_type = other DNA
caagcagaagacggcatacgagatatcagtgtgactggagttcagacgtgtgctcttccgatct

SEQ ID NO: 34 - DNA, 64 bp, synthetic construct
  misc_feature 1..64: note = synthetic construct
  source 1..64: mol_type = other DNA
caagcagaagacggcatacgagataggaatgtgactggagttcagacgtgtgctcttccgatct

SEQ ID NO: 35 - DNA, 12 bp, synthetic construct
  misc_feature 1..12: note = synthetic construct
  source 1..12: mol_type = other DNA
tgcgncgaaccc

SEQ ID NO: 36 - DNA, 72 bp, synthetic construct
  misc_feature 1..72: note = synthetic construct
  source 1..72: mol_type = other DNA
gctggggaactacaggctgacagtccgggcggtaaagcgccgaacccacggtacagtttgagttctggttct